(19) United States
(12) Patent Application Publication
(10) Pub. No.: US 2026/0369356 A1
(43) Pub. Date: Nov. 19, 2026

(54) SYSTEMS AND METHODS FOR DETERMINING LINKAGE OF SEQUENCE READS ON A FLOW CELL

(71) Applicant: Illumina, Inc., San Diego, CA (US)

(72) Inventors: Taher Mun, West Chester, PA (US); James Han, San Carlos, CA (US); Mitchell A. Bekritsky, Decatur, GA (US); Vitor Ferreira Onuchic, San Diego, CA (US); Niall Anthony Gormley, Cambridge (GB); Jacqueline Weir, Cambridge (GB); Jonathan Mark Boutell, Bishops Stortford (GB); Louise Fraser, Great Cambourne (GB); Maria Ines Martins Vitoriano, Baldock (GB)

(21) Appl. No.: 19/493,002

(22) PCT Filed: Sep. 10, 2024
(86) PCT No.: PCT/US2024/045996

§ 371 Date: Dec. 12, 2025

Related U.S. Application Data
(60) Provisional application No. 63/582,092, filed on Sep. 12, 2023.
(60) Provisional application No. 63/610,279, filed on Dec. 14, 2023.
(60) Provisional application No. 63/565,255, filed on Mar. 14, 2024.

(51) Int. Cl. G16B 30/10; G16B 40/00

(52) U.S. Cl. CPC G16B 30/10; G16B 40/00

(57) ABSTRACT

Described are DNA sequencing systems and methods. Systems and methods may calculate a genomic null distribution of read pairs on a flow cell having approximately zero probability of being linked with one another and calculate a spatial null distribution of read pairs having approximately zero probability of being spatially linked. The system may generate a link quality score representing a probability of incorrectly assigning a link between the two polynucleotide reads based on the calculated genomic null distribution and the calculated spatial null distribution.

### CROSS-REFERENCE TO RELATED APPLICATIONS

This application claims priority to U.S. Provisional Application Nos. 63/582,092, filed Sep. 12, 2023, and 63/565,255, filed Mar. 14, 2024, the contents of which are incorporated by reference in their entirety.

### BACKGROUND

### Field

The present disclosure relates to DNA sequencing systems and methods. In particular, this disclosure relates to systems and methods for determining and improving the number of links, the percentage of links and the quality of links between pairs of reads on a sequencing flow cell.

### Background

Genomic DNA is often too long to be directly sequenced using modern sequencing technologies. Library preparation is a step performed before genome sequencing to facilitate the sequencing process and ensure accurate and efficient analysis of the genomic DNA. Library preparation involves fragmenting the DNA into smaller, manageable pieces. This fragmentation can be achieved through physical or enzymatic methods. Fragmented DNA allows for more efficient sequencing and enables the reconstruction of the original genome during data analysis. Library preparation also involves attaching adapter sequences to the fragmented DNA. Adapters contain specific sequences that are recognized by the sequencing platforms and are used for sequencing the DNA fragments. These adapters provide priming sites and identification tags for the sequencing process.

Traditional nucleic acid sequencing methods, and several types of next-generation sequencing methods, use a shotgun approach to sequence large genomic DNA fragments, called template nucleic acid molecules. Specifically, template nucleic acid molecules are first fragmented in solution into smaller pieces that are amenable to next-generation sequencing methods on a flow cell. One of the difficulties of this approach is that by the time the smaller sequence fragments from the template genomic sequences have been read, knowledge of their connectivity and proximity to each other in the original template genomic sequence is lost. In order to accurately map genomic regions, traditional nucleic acid sequencing methods will typically sequence a specific region (or the genome) a number of times during the sequencing process.

Next-generation sequencing approaches will sequence every base in a sample several times. This is for several reasons. First, having multiple observations per base results in a more reliable determination of the correct sequence of a nucleic acid. In addition, some reads are not distributed evenly over an entire genome, simply because the reads will sample the genome in a random and independent manner. Therefore, many bases will be covered by fewer reads than the average coverage, while other bases will be covered by more reads than average. This coverage is expressed by a coverage metric, which is the number of times a sample genome has been sequenced, termed the “depth” of sequencing coverage. For applications where you aim to sequence only a defined subset of an entire genome, like targeted resequencing or RNA sequencing, coverage means the number of times you sequence that subset. For example, for targeted resequencing, coverage means the number of times the targeted subset of the genome is sequenced.

The depth of sequencing coverage often determines whether variant discovery can be made with a certain degree of confidence at particular base positions. Sequencing coverage requirements vary by application, but in general, at higher depths of coverage, each base is covered by a greater number of aligned sequence reads, and so base calls can be made with a higher degree of confidence. To attain high coverage in genomic sequencing, researchers would typically increase the read depth, which involves generating a greater number of sequencing reads for a given sample. This can be achieved by either sequencing more DNA molecules or by sequencing the target nucleic acid additional times to determine the proper sequence information. Additionally, the library preparation process is typically optimized to minimize DNA loss and bias, ensuring that a larger proportion of the original DNA is successfully sequenced.

### SUMMARY

One embodiment is a system for sequencing a polynucleotide comprising at least one processor, and a non-transitory computer readable medium comprising instructions that, when executed by the at least one processor, cause the system to: retrieve data comprising polynucleotide sequence reads and their spatial location on a flow cell; wherein the flow cell comprises clusters of amplified nucleic acid fragments of a polynucleotide, and wherein a probability of the clusters being located near each other on the flow cell is correlated with a genomic distance between the nucleic acid fragments of the polynucleotide and wherein each cluster provides one or more of the sequence reads; determine links between sequence reads originating from the same polynucleotide molecule; and store information on links between sequence reads in a computer memory, wherein at least one of: (a) the linked sequence reads have a link quality score of at least 20 on a Phred scale, and the linked sequence coverage has an average depth of at least 30-fold (30×) of a reference genome; (b) the percentage of sequence reads on the flow cell with a determined link to at least one other sequence read is greater than 45% and the average sequence coverage is at least 70× coverage for all sequence reads; (c) the percentage of sequence reads on the flow cell with a determined link to at least one other sequence read is greater than 35%, and the average sequence coverage is at least 90× coverage for all sequence reads; and (d) the percentage of sequence reads on the flow cell with a determined link to at least one other sequence read is greater than 25% and the average sequence coverage is at least 120× coverage for all sequence reads.

Yet another embodiment is a method for sequencing a polynucleotide, comprising: obtaining sequence reads from a flow cell, wherein the flow cell comprises clusters of amplified nucleic acid fragments of the polynucleotide, and wherein a probability of the clusters being located near each other on the flow cell is correlated with a genomic distance between the nucleic acid fragments of the polynucleotide and wherein each cluster provides one or more of the sequence reads; and determining links between sequence reads originating from the same polynucleotide molecule, wherein the at least one of: (a) the linked sequence reads have a link quality score of at least 20 on a Phred scale, and the linked sequence coverage has an average depth of at least 30-fold (30×) coverage of a reference genome; (b) the percentage of sequence reads on the flow cell with a determined link to at least one other sequence read is greater than 45%, and the average sequence coverage is at least 70× coverage for all sequence reads; (c) the percentage of sequence reads on the flow cell with a determined link to at least one other sequence read is greater than 35%, and the average sequence coverage is at least 90× coverage for all sequence reads; and the percentage of sequence reads on the flow cell with a determined link to at least one other sequence read is greater than 25%, and the average sequence coverage is at least 120X coverage for all sequence reads.

In some aspects, the techniques described herein relate to a method of generating a link quality score, including calculating a genomic null distribution of a plurality of read pairs on a flow cell having approximately zero probability of being linked with one another; calculating a spatial null distribution of a plurality of read pairs having approximately zero probability of being spatially linked with one another; and generating a link quality score representing a probability of incorrectly assigning a link between the two polynucleotide reads based on the calculated genomic null distribution and the calculated spatial null distribution.

In some embodiments, a method is provided for generating a link quality score, the method including calculating a genomic null distribution of a plurality of read pairs on a flow cell having approximately zero probability of being linked with one another; calculating a spatial null distribution of a plurality of read pairs having approximately zero probability of being spatially linked with one another; and generating a link quality score representing a probability of incorrectly assigning a link between the two polynucleotide reads based on the calculated genomic null distribution and the calculated spatial null distribution.

In some embodiments, the method includes calculating the genomic null distribution of the plurality of read pairs includes determining read pairs from clusters on the flow cell. In some embodiments, calculating the genomic null distribution of the plurality of read pairs includes determining read pairs from clusters that are spatially distant from one another on the same or opposite surfaces of a flow cell. In some embodiments, the clusters used for calculating the null distribution are on the same surface but opposite ends of the flow cell. In some embodiments, the clusters used for calculating the null distribution are opposite surfaces of the flow cell.

In some embodiments, the method includes calculating the spatial null distribution of the plurality of the plurality of read pairs includes determining read pairs from clusters on the flow cell. In some embodiments, the techniques described herein relate to a method, wherein a first cluster has nucleic acid derived from a first chromosome and a second cluster has nucleic acid from a second, different chromosome.

In some embodiments, a method may further include applying threshold levels for spatial distance and genomic distance to the plurality of read pairs on the flow cell.

In some embodiments, a method may further include determining at least one threshold level such that read pairs from clusters derive from the same DNA molecule with a predetermined probability for spatial distance and genomic distance using the quality of the link. In some embodiments, a method may further include estimating a linking rate for the total links between read pairs on the flow cell for the threshold levels.

In some embodiments, the method may further include selecting a threshold level based on the link quality score. In some embodiments, the method may further include adjusting an assay parameter based on the link quality score. In some embodiments, the assay parameter includes at least one of flow cell design, coverage, DNA loading density, DNA loading method, DNA loading strategy, the intensity of an electric field applied to a flow cell, linearity of the flow of on the flow cell, the 3D structure of the DNA, a solution applied to the flow cell, and indexing. Similarly, in some embodiments, the assay parameter of flow cell design includes at least one of pitch size, well depth, and flow cell size.

In some embodiments, the techniques described herein relate to a method, wherein generating a link quality score includes generating a false positive rate. In some embodiments, the false positive rate is proportional to the product of the percentage of genomic null links and the percentage of spatial null links. In some embodiments, generating a link quality score includes generating an expected percentage of genomic-null links and spatial-null links for links from the plurality of read pairs on the flow cell below the threshold levels for spatial and genomic distance.

In some embodiments, the expected percentage of genomic-null links and spatial-null links are proportional to the product of the false positive rate and the number of links from the plurality of read pairs on the flow cell below the threshold levels for spatial distance and genomic distance. In some embodiments, generating a link quality score includes generating a false discovery rate. In some embodiments, the false discovery rate is proportional to an estimated number of false links divided by the total number of links in the plurality of read pairs on the flow cell. In some embodiments, the link quality score is represented using the Phred scale.

In some embodiments, the method relates to calculating a genomic null distribution and spatial null distribution of read pairs is performed on a subset of the plurality of read pairs on a flow cell. In some embodiments, the read pairs are representative of the total read pairs on the flow cell. In some embodiments, the quality of a link between the two polynucleotides represents the quality of the links for the plurality of reads on the flow cell below the threshold levels for spatial distance and genomic distance. In some embodiments, the quality of a link between the two polynucleotides is applied to individual read pairs within the plurality of reads on the flow cell.

In some embodiments, a method may further include assigning a candidate link between two polynucleotide reads using the spatial positional data. In some embodiments, assigning a candidate link between two polynucleotide reads using the spatial positional data includes determining a link quality score that a first cluster and a second cluster on sequencing flow cell originate from the same target polynucleotide. In some embodiments, generating a link quality score includes a statistical test including at least one of a t-test, a chi-square test, a permutation test, and a deconvolution analysis.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear. While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

FIG. 1A schematically illustrates a non-limiting example of a solid support which can be used to perform embodiments of the disclosed sequencing technology.

FIG. 1B schematically illustrates a non-limiting example of template molecules on a flow cell.

FIG. 2 shows a flowchart of an example method for sequencing reads with a high link rate between the sequence reads.

FIG. 3 shows a flowchart of an example method for generating a link quality score.

FIG. 4 shows an overview of a method for estimating the percentage link rate.

FIG. 5A shows a bar chart demonstrating the difference in link rates for processing conditions for a sequencing system at two concentrations of input DNA each. FIG. 5B shows a line graph depicting the relationship between deduped linked read coverage and the combined false positive (FP) plus false negative (FN) rates for germline detection.

FIG. 6 shows Panel A and Panel B bar charts reporting experimental results of link rates performed with crude samples of patients' blood. In Panel A, white blood cell count (WBC) per blood sample is shown as dots. In Panel B, nanograms of genomic DNA in 25 μl of blood is shown as dots.

FIG. 7, Panels A and B show two line plots of calculated link quality Q scores for two different experimental systems by using false positive rates.

FIG. 8 shows Panels A-F of a schematic of a random flow cell (Panel A), a patterned flow cell (Panel B), the center to center pitch of flow cells (Panel C), the decreased center to center pitch of flow cells based on feature size (Panel D), an electron micrograph image of a cross-sectional side view of flow cell wells (Panel E) and a top down electron micrograph image of flow cell wells (Panel F).

FIG. 9 illustrates post-sequencing spatial locations of exemplary sequencing reads on a solid support according to some embodiments.

FIG. 10 illustrates non-limiting examples of metrics related to spatial distance information from the clusters according to some embodiments.

FIG. 11 shows a flowchart of an example method for generating a link quality score.

FIG. 12 schematically illustrates a system including a memory comprising a sequencing module for achieving high link rates.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

This disclosure describes performing sequencing methods using improved assays that result in a relatively high rate of links between sequence reads. A link between two sequence reads on a flow cell indicates that those two reads are found proximal to each other on the original template nucleic acid molecule from which the reads were derived prior to fragmenting. Thus, the reads that are “linked” to one another will map near each other on the original template nucleic acid molecule. Determining links includes analyzing sequence reads that are located spatially near each other on a flow cell. This relates to the discovery that when template nucleic acid molecules, such as genomic DNA, are fragmented on a flow cell, it is more likely that sequences located spatially near each other on the flow cell were derived from the same template nucleic acid molecule. A first sequence located near a second sequence may be assigned a link between the two sequences if they are found to be near each other on the original template, which is useful for a proper alignment and assembly of the sequenced nucleic acid molecules. As there is some random chance that two unrelated sequences end up near each other on the flow cell, a link quality score may be assigned to links in order to determine, and potentially filter, true links between sequence reads from spurious links. The link quality score is also useful for determining whether a sequencing system and method generated more or fewer true links between sequences.

The disclosure describes methods and systems which can improve the rate of generating links between sequence reads, and also provides for systems and methods that increase the rate of creating links between sequences by performing various assays to increase link rates. A number of techniques for improving the number of sequence read links on a flow cell may include library preparation techniques which result in a flow cell having an increased number of linked reads as compared to a flow cell having sequence reads which were not subjected to the library preparation techniques. For example, some techniques which were discovered to improve the number of links between sequence reads may include, as non-limiting examples, low concentration loading, multi-flush loading methods, using a crude lysate, using high molecular weight DNA extraction techniques, using additives and optimizing flow rates. These techniques will be discussed in more detail in the following sections.

The disclosure provides for systems and methods for sequencing nucleic acids using improved assays, such as polynucleotides, with an improved link rate between sequence reads on the surface of the flow cell. The typical procedures used to prepare a library for sequencing short reads are not necessarily optimal procedures for generating linked reads. The methods traditionally used for preparing libraries and clustering on instruments, which are used for short read sequencing, may not be the most effective when the objective is to produce linked reads. Linked reads, which offer more comprehensive genomic insights by preserving information about longer DNA fragments, may use a consistent sequencing process, while the preparatory stages and clustering techniques may be adapted to meet the specific objectives of linked read sequencing. Various assay parameters have been adjusted to increase the link rate. For example, it has been discovered that adding a lower concentration of DNA on the flow cell increases the number of true links on the flow cell. This result can be explained where the lower concentration of DNA added to the flow cell results in less crowding of unrelated DNA on the flow cell prior to fragmentation, which in turn would result in less overlap of unrelated fragments on the flow cell. A lower rate of spurious links allows for higher thresholds of genomic and spatial distances, which are criteria for establishing a link with a high quality. Accordingly, the total amount of DNA on the flow cell might be reduced but the rate of links between sequences will increase. Crude lysates, which are non-extracted and simply lysed samples from sources like blood or other sources, were found to yield higher link rates compared to samples where DNA is extracted in a standard way. This enhanced performance is attributed to the high molecular weight of DNA in these crude lysates. Similarly, high molecular weight extraction kits or gentle sample handling can help generate conditions for better and more spatial links in total or on a percentage basis.

The benefit of linked reads may be partially quantified by determining the number of false positives and false negatives in the analysis of a truth set. Based on an empirical analysis increasing the link rates in sequencing data results in diminishing returns in performance gain for identifying false positives and negatives once around a depth of approximately 30× linked coverage may be achieved. Thus, each base should, on average, be covered by 30 different reads on a flow cell. Experiments were performed to determine the effect of coverage depth on the reduction of false positives (FP) and false negatives (FN) in germline variant calling. It was observed that the combined FP+FN decreases as the deduped linked depth of read coverage increases, indicating that higher depths of coverage generally lead to fewer errors in variant calling. However, the performance gains start to plateau once approximately 30× coverage depth is reached. Similarly, percentage reductions in FP+FN when increasing coverage depth from 8× to 16×, 16× to 24×, 24× to 32×, and 32× to 40× showed the highest reduction observed when going from 8× to 16× of depth of coverage.

Some embodiments relate to providing a link quality of at least a Phred score of 20. The Phred quality score is a common metric used in sequencing to indicate the quality of nucleotide identification. This score is logarithmically linked to the error probability. For instance, a Phred score of 20 corresponds to an error probability of 1%, a score of 30 to 0.1%, and so on. In the context of a metric like “link quality” on the Phred scale, determining a lower threshold depends on the specific requirements of the sequencing project. However, as a general guideline: A Phred score of 20 is traditionally considered the minimum for reliable data, equating to a 99% confidence level. In some embodiments, the link rate of linked reads with a linked quality score of 20 is about 45-50% at 70× depth of coverage. This equates to approximately 30× depth coverage of the linked data.

The disclosure also provides for systems and methods for establishing the quality of a link between two or more read pairs on a flow cell. The “link” as discussed herein is the probability that two pairs of reads on a sequencing flow cell are derived from the same original nucleic acid molecule. In some next generation sequencing (NGS) systems, fragments of long DNA, such as genomic DNA, from a biological source are fragmented to create shorter fragments which can be sequenced in a single read. The fragmentation process will create these shorter fragments which land on the flow cell, and the spatial location of each fragment may be related to the original nucleic acid molecule from which the fragment was derived. For example, fragments which come from the same nucleic acid molecule have been found to bind closer together on the flow cell as compared to fragments which come from different original nucleic acid molecules. Accordingly, if two clusters of reads on a flow cell are close together spatially and also close together on an individual's genome, the clusters are more likely to have come from the same nucleic acid molecule. However, unrelated fragments may also bind to the flow cell near one another, which leads to an uncertainty in the probability that adjacent clusters originate from the same molecule. A number of factors could affect the probability that unrelated clusters would land in a similar area, and these factors may change based on a variety of experimental conditions, as described herein. Embodiments of the disclosure provide a statistical method for calculating the probability that two reads are linked, such that on a flow cell the two reads were derived from the same nucleic acid molecule.

Embodiments of the disclosure relate to systems and methods for sequencing target nucleic acids by fragmenting the target nucleic acid and distributing the fragments onto a flow cell. As the fragments are distributed along the flow cell, they bind capture primers and are then used to create clusters by well-known technologies, such as those provided by Illumina Inc. (San Diego, CA). As described above, according to the methods of this disclosure, fragments which were derived from the same template genomic sequence are more likely to bind to the flow cell in spatially nearby positions as compared to fragments that are from different template genomic sequences, particularly when the fragmentation is performed directly on the flow cell using immobilized transposome complexes on the surface of the flow cell. In regular library preps with fragmentation happening prior to loading, fragments can land anywhere in the flow cell independently of whether they came from the same molecule. However, when fragmentation is performed directly on the flow cell spatial information is retained. This spatial information can be used to help guide assembly and variant calling of the original template genomic sequence, as will be described in more detail below.

For example, one embodiment is a method for assigning nucleic acid sequence reads to target polynucleotides, which includes providing transposome complexes. In some embodiments, the transposome complexes include a transposase and a first polynucleotide having end sequences which can be used to fragment the target polynucleotides and insert into each fragment an end sequence or tag which can be used to bind to capture probes located on the substrate. The method can include contacting the transposome complexes, which are bound to a solid substrate, with the target polynucleotides under conditions to fragment the target polynucleotides and may add capture sequences to the ends of each fragment.

Once the fragments have been bound to a substrate, the bound fragments can be amplified to form a plurality of nucleic acid clusters on the substrate. The location of each cluster on the flow cell can then be determined before, during or after performing sequencing by synthesis reactions (SBS) to obtain the nucleotide sequence of each fragment located in each cluster. Once the nucleotide sequence of each cluster has been determined, the method can start to map those reads to determine the original target polynucleotide from which the read originated. In some embodiments, the mapping process takes into account the spatial location of each cluster, such that clusters which are closer to each other on the flow cell are more likely to have originated from the same target polynucleotide. In some embodiments, the library preparation steps are performed on the flow cell, which may reduce the complexity and the amount of equipment associated with the systems. Furthermore, by mapping the sequenced fragments to target polynucleotides using the spatial information accompanying each cluster, the method performs more accurate mapping operations as compared to methods that do not take the spatial location of each cluster into account during the mapping process. Therefore, spatial information that includes relative distances between various clusters on a flow cell may be leveraged to adjust mapping information, thereby increasing the read quality of previously identified multi-mapped reads. In the past, identified multi-mapped reads may have been discarded. Increasing the read quality of these previously discarded reads, by improving the confidence of read pair's alignment based on linking information with a high link quality score, may improve the alignment information and quality of information used in certain genomic analysis applications including, but not limited to, variant calling.

Processing DNA samples suitable for high-throughput sequencing that retain information on the original configuration of the DNA samples provides useful information on co-located fragments. However, co-located fragments will not always originate from the same molecule, which raises some difficulty in terms of defining the confidence that any two co-located fragments originated from the same molecule. The ambiguity between real and spurious links may limit the applicability of using the spatial information, or for integrating the linked reads into mapping or alignment processes. Accordingly, assessing and quantifying the relationship of fragments on a flow cell can have a significant impact on mapping, alignment, and variant calling. An ability to filter out spurious links between reads, as well as quantifying high quality links and resulting alignments of reads impacts processing requirements. By increasing the quality of linked reads, the methods of the disclosure also serve to reduce the memory footprint of sequencing processes. High-quality linked reads provide more accurate and reliable genomic information. This means that fewer reads are needed to confidently cover and assemble a particular region of the genome. In lower-quality sequencing, by contrast, more reads (and hence more data) are often necessary to achieve the same level of confidence, leading to data redundancy. By reducing the number of redundant reads, the overall volume of data that needs to be stored and processed is decreased, thereby lowering the memory requirements. With high-quality linked reads, the computational processes involved in sequencing, such as alignment and assembly, can be more accurate in difficult to map regions of the genome. High-quality reads align more accurately and with less computational effort to reference genomes. This means that less computational power and memory are required for these processes. Additionally, high-quality data can reduce the need for extensive error correction and data cleaning steps, which are resource-intensive tasks.

High-quality linked reads reduce the need for extensive data cleaning and filtering processes that are necessary to deal with errors and ambiguities in lower-quality data. These processes not only consume computational resources but also increase the memory footprint by generating additional intermediate data files. With increased read quality, it is possible to more accurately map and align polynucleotide sequences in difficult regions of the genome.

Described herein are systems and methods of establishing link quality scores for the links determined between read pairs based on spatial information obtained on the flow cell, This spatial information may be, for example, the cartesian coordinates of the cluster which contains a particular read on the flow cell. The spatial information may include a location of a well on a substrate in one embodiment. To establish these spatial links between two reads, in one embodiment two thresholds are used. The first is the spatial distance threshold, which represents the physical distance between two reads on the flow cell.

In some embodiments, the spatial distance may be measured in nanometers. In some embodiments, the spatial distance may be measured in a unit of length relative to the flow cell. For example, a flow cell unit may be relative to the size and/or spacing of patterned clusters on a flow cell. In some embodiments, two differently patterned flow cells may have different absolute units of length due to different density of clusters on the surface. In some embodiments, the spatial distance may be an absolute unit of length, or any other unit of length consistent with the disclosure. In some embodiments, the spatial distance may be included in a FASTQ file, which generally is a text file that contains the sequence data from the clusters that pass filter on a flow cell. FASTQ files can be used as sequence input for alignment and other secondary analysis software.

The second threshold is the genomic distance threshold, representing the distance between the two reads on the genome after mapping to a reference genome. An empirical method for establishing thresholds will vary widely between experimental conditions. This disclosure provides for methods to attach a link quality score to a link as a factor of the spatial and genomic distance between two potentially linked reads. As described in more detail below, one method of determining the quality of a link between two reads is to estimate the null distribution of pairwise read pairs. This null distribution can provide the basis for calculating the “false discovery rate”, which can then be used as a proxy for the link quality score of the link.

A linking quality score is defined as a numerical representation that quantifies the reliability of a link between two read pairs. This score may be calculated using multiple metrics that contribute to the quality of the link, and the linking quality score may serve as a composite measure that simplifies complex relationships into a single value.

A linking quality score may provide a basis for comparison or decision-making. For example, a high linking quality score between two read pairs might indicate that two reads are highly likely to originate from the same DNA fragment, and thus should be paired for further analysis, but also that the conditions used to generate that link may be tuned and evaluated on the basis of the score. The formula for calculating a linking quality score may vary and may be determined based on a false discovery rate, a metric quantifying type II error (false negative errors), statistical or mathematical model, a weighted average of different contributing metrics, and/or a machine learning model trained to predict link quality based on multiple features. By way of nonlimiting examples, the following metrics may contribute to evaluating the quality score for linked reads on a flow cell. In some embodiments, read length and quality may be useful because longer reads with high base quality scores may be more likely to yield accurate linking information, which in turn is beneficial for genome assembly and variant calling. In some embodiments, coverage depth and uniformity may also help ensure that genomic regions are sequenced adequately, with uniform coverage for difficult-to-sequence regions. In some embodiments, phasing efficiency, which measures how accurately linked reads are assigned to their respective haplotypes, in, for example, a training set, may also affect the precision of haplotyping analyses. In these cases, the linking quality score aims to encapsulate diverse considerations into a single number representing a link's overall “quality,” thereby facilitating quantitative analysis.

A relevant aspect to consider is the relationship between the surface area in the flow cell and the likelihood of having two read fragments land close to each other by chance. A small surface area in the flow cell reduces the probability of two read fragments landing in close proximity due to the limited surface area to accommodate reads. Conversely, a larger surface area in the flow cell increases the likelihood of chance occurrences where read fragments, including those from different chromosomes, land in close proximity. Consequently, utilizing a large threshold distance for read fragments in the flow cell to establish spatial links, in turn leads to an increased likelihood of considering spurious links. The larger the area under consideration, the information gain from the sequencing process potentially diminishes, especially when maintaining a minimum link quality threshold. The link quality score communicates whether the link is spurious or not, and downstream methods may determine which quality scores to consider. While there may still be links within a larger surface area, distinguishing true links from spurious links poses additional challenges.

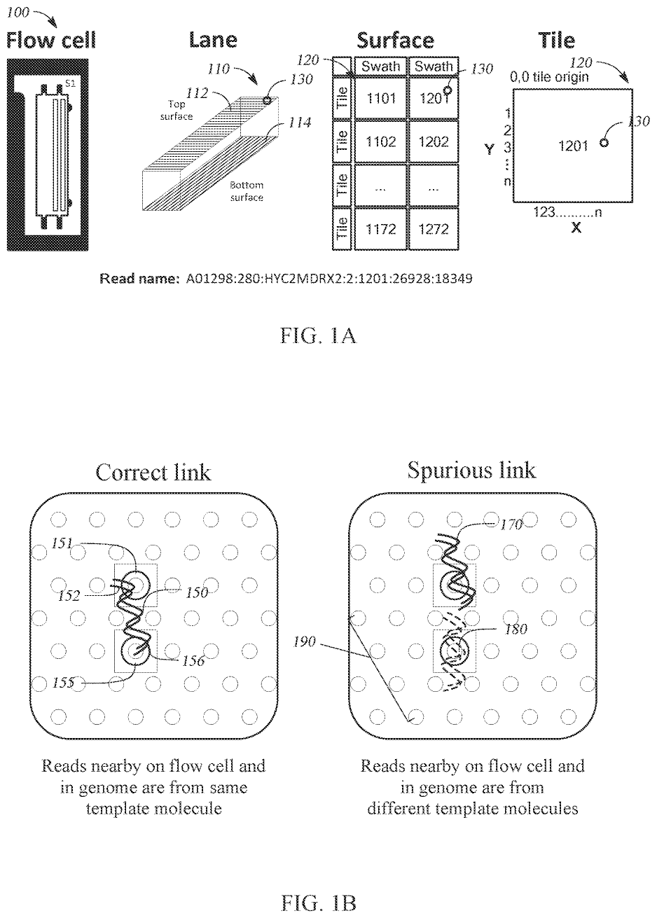

Referring now to FIG. 1A, in some embodiments, a flow cell 100 that provides spatial information of read pairs may include a plurality of lanes 110. Each lane 110 includes a plurality of surfaces. As shown, in some embodiments of the flow cell 100, a lane includes a top surface 112 and a bottom surface 114. If the reads being compared are on opposite surfaces, the distance between them is considered infinite because the assumption is that they cannot be linked. In some embodiments, each surface is subdivided into a plurality of tiles 120. As shown, a cluster 130 may be located on a tile 120 that is designated as 1201. This designation serves as an illustrative example only and is not limited to the alphanumeric characters shown in the figure. In some embodiments, the tile 120 includes two-dimensional X-Y coordinates as shown to provide the spatial information between clusters. In some embodiments, the X-Y coordinates may be derived from information stored in a FASTQ file. In some embodiments, X-Y coordinates may be stored in or derived from a BCL (Base Call) file, which is a binary file format commonly associated with next-generation sequencing (NGS) platforms.

In some embodiments, the subdivision of the surface into tiles 120 is an artificial separation so that the surface of the flow cell is not separated into physical tiles, but instead the images captured by a camera can be segmented into tiles. As shown, the tiles 120 are subdivided into swaths, which roughly correspond to the pixel width of a camera used to capture images of the flow cell. In some embodiments, the tile 120 denotes the size of an image that can be captured by the camera. In some embodiments, the X-Y coordinates are pixel values. In some embodiments, 1 unit of a tile 120 can be approximated to be 1/10^{th} of a pixel. A physical separation is contemplated in some embodiments where the tile can have physical barriers, wells, and other structures that separate one portion of the flow cell from another portion of the flow cell. In some embodiments, spatial information, including X-Y coordinates, for clusters such as cluster 130 are obtained by a camera that processes the pixel value of the digital image.

An example of an assay, and/or associated library preparation, that may provide spatial information on sequenced reads may be an assay that is performed on a substrate having transposome complexes immobilized thereon. The transposome complex may, for example, include a transposase and a first polynucleotide including an end sequence and a first tag in some embodiments. The assay may proceed by contacting the transposome complexes with target polynucleotides under conditions to fragment the target polynucleotides. The fragmented target polynucleotides may then be amplified to form a plurality of nucleic acid clusters on the substrate. The plurality of nucleic acid clusters on the substrate are microscopically observable and their location data may be recorded. After the location information has been obtained, then the nucleic acid sequence reads of the fragmented nucleic acids may be sequenced and the corresponding location data may be stored.

In some embodiments, spatial information may be used to link reads together, where the link between the reads can be a physical or non-physical link. In some embodiments, spatial information is used to link reads together to form a longer linked read with one or more read subpairs. The linked read pairs have expected properties such as, but not limited to, expected length distribution, distance between pairs, and number of pairs. These properties can be leveraged in genome analysis. Referring now to FIG. 1B, in one non-limiting example, a flow cell 148 contains a set of clusters, with each cluster comprised of nucleic acid fragments. A Fragment 1 is located in a cluster 151 and was derived from a segment 152 on an original nucleic acid 150. Similarly, a Fragment 2 is located in an adjacent cluster 155 and was derived from a segment 156 on the original nucleic acid 150. Fragment 1 and Fragment 2 may be linked using spatial information that confirms Fragments 1 and 2 are from the same original nucleic acid 150 or any other polynucleotide consistent with the disclosure. The length of the linked read construct connecting these fragments may be denoted as the length of Fragment 1 and Fragment 2 plus, for example, 2 units. Therefore, in that example, the distance between Fragments 1 and 2 is 2 units, such as 2 units of length on the flow cell. Note that any arbitrary unit may be used, such as X Y coordinates or flow cell coordinates, shown as distance 190 as 4 flow cell units. In situations where the clusters 151 and 155 are in close packed arrays, a conversion may be used to convert flow cell units to X-Y coordinates and vice versa. In another non-limiting example, a flow cell 160 may contain a Fragment 3 and a Fragment 4 which might be spuriously tagged as a link. In this example, Fragment 3 may have originated from an original nucleic acid 170, while Fragment 4 may have originated from a different original nucleic acid 180. In another non-limiting example, Fragment 3, Fragment 4 and a Fragment 5 may be linked together using spatial information to confirm that Fragments 3, 4, and 5 were derived from the same polynucleotide molecule. The distance between Fragments 3 and 4 in this example may be 5 units or less and the distance between Fragments 4 and 5 may be 5 units or less.

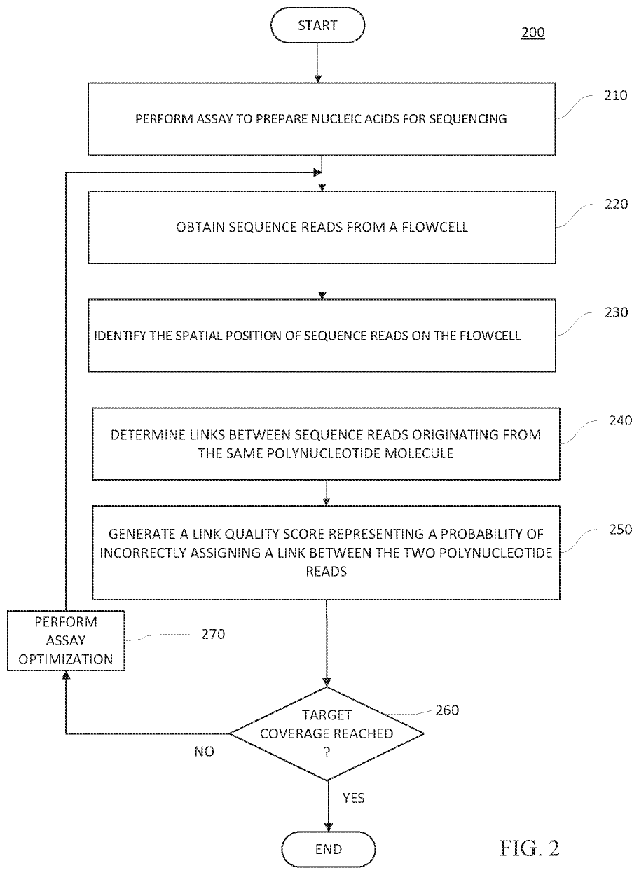

FIG. 2 is a flowchart of an example method 200 for sequencing a polynucleotide that achieves a desired link rate between reads. At step 210, an assay is performed to prepare nucleic acids, such as DNA or RNA, for sequencing. This preparation typically involves procedures such as extraction, quantification, and fragmentation of the nucleic acids, ensuring they are in the proper form and concentration for subsequent analysis. In methods described herein, fragmentation may occur after the DNA or RNA is first bound to the flow cell.

Following the preparation, the workflow progresses to step 220, where sequence reads are obtained from a flow cell. As described herein, a flow cell may include clusters of amplified nucleic acid fragments of the polynucleotide, and when performing methods of the disclosure the probability of the clusters being located near each other on the flow cell is correlated with a genomic distance between the nucleic acid fragments of the polynucleotide. In some embodiments, each cluster provides one of the sequence reads.

At step 230, the method identifies the spatial position of sequence reads on the flow cell. Accurate identification of where each read is located on the flow cell may be embedded in the images taken of the flow cell surface, where a cluster may correspond to a particular pixel in an image. Methods of converting from pixels to flow cell units or other measures of distance are described further herein.

Next, at step 240, the method may proceed by determining links between sequence reads obtained in the prior step, where the links would indicate that the sequences originated from the same polynucleotide molecule. Consistent with the disclosure, various parameters may be tuned in order to consistently achieve a high link rate between the sequence reads. However, the sequence depth, coverage and link rate have some tradeoffs. For example, as described earlier, in order to increase coverage, a researcher might typically increase the loading of sample onto a flow cell to generate more reads, but this strategy could lead to a lower link rate. The following non-limiting parameters demonstrate high link rates and high coverage and are achievable following the methods provided by the disclosure. Note, however, in some cases, increasing from 0.5× to 1× coverage may increase the link rate, as when approaching full occupancy of the flow cell the additional coverage may begin to drop. As an additional example, crude lysates have been shown to achieve both high coverage and high link rates with a range of sample inputs.

To optimize link rates in sequencing, several strategies and parameters may be adjusted: lowering the concentration of the sample, as has been demonstrated in datasets described herein; utilizing crude lysate, which may affect the link rates depending on its processing level; performing high molecular weight (HMW) DNA extraction to preserve larger fragments of DNA; implementing a multi-flush technique, which could involve multiple sample flows to optimize the sample environment; adding specific additives to stabilize DNA or improve reaction conditions; optimizing the flow rates within the sequencing apparatus to influence the movement and capture of DNA; ensuring gentle sample handling to maintain DNA integrity; employing molecular combing to stretch DNA molecules, which could enhance the clarity of spatial relationships between segments; and designing active flow cells and microchannels to facilitate efficient link capture. Additionally, multiple sample flows could also enable repeatedly adding low concentration of template to maintain high link rate, but also high coverage. These methods, along with considerations for flow cell design, DNA loading density, method, and strategy, are useful to enhancing link rates and thus the quality of sequencing data.

The workflow then assesses the quality of these links at step 250 by generating a link quality score. In some embodiments, the link quality score may be the Phred-scaled log likelihood that the two reads are not linked, given their flow cell coordinates (e.g. cartesian coordinates) and genomic coordinates. In some embodiments, this score correlates with the probability of a link between two polynucleotide reads being correctly linked. The process reaches a decision point at step 260, where it is determined whether the target coverage has been reached. In some embodiments, if the coverage is insufficient, the workflow may optionally loop back to step 270, where assay optimization is optionally performed to further improve the sequencing output.

The sequence of steps concludes once the target coverage is confirmed to have been reached, signifying the end of the experimental workflow and the readiness of the data for downstream analysis. In some embodiments, the linked sequence reads have a link quality score of at least 20, and the linked sequence coverage is an average of at least 30× coverage of linked sequence reads. In some embodiments, the linked sequence reads have link quality score of at least 25, and the linked sequence coverage is an average of at least 30× coverage of linked sequence reads. In some embodiments, the percentage of sequence reads on the flow cell with a non-spurious link to at least one other sequence read is greater than 45% and the average sequence coverage is at least 70× coverage for all sequence reads. In some embodiments, the percentage of sequence reads on the flow cell with a non-spurious link to at least one other sequence read is greater than 35% and the average sequence coverage is at least 90× coverage for all sequence reads. In some embodiments, the percentage of sequence reads on the flow cell with a non-spurious link to at least one other sequence read is greater than 25% and the average sequence coverage is at least 120× coverage for all sequence reads.

### Methods of Calculating a Quality Score

In some embodiments, a method is provided for generating a link quality score, the method including calculating a genomic null distribution of a plurality of read pairs on a flow cell having approximately zero probability of being linked with one another; calculating a spatial null distribution of a plurality of read pairs having approximately zero probability of being spatially linked with one another; and generating a link quality score representing a probability of incorrectly assigning a link between the two polynucleotide reads based on the calculated genomic null distribution and the calculated spatial null distribution.

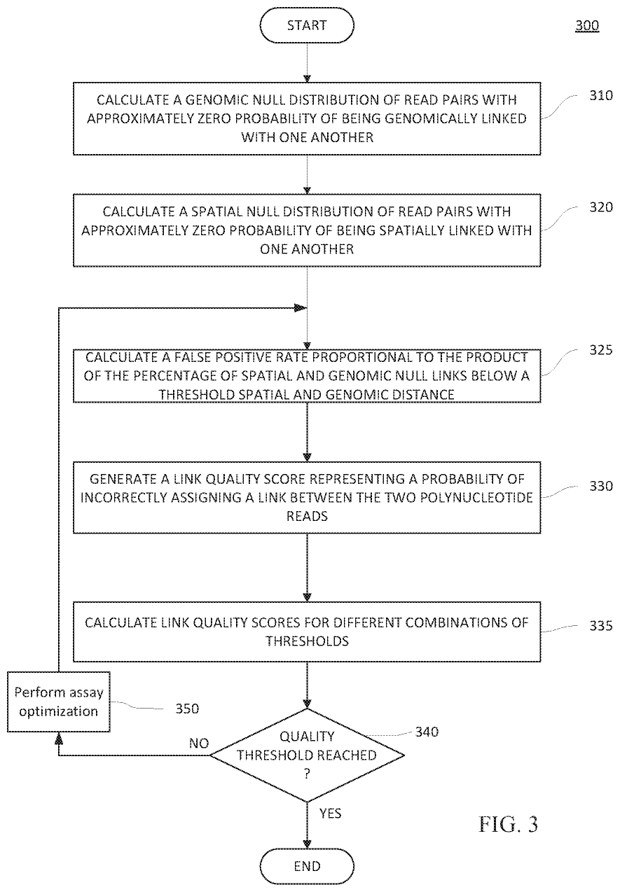

The disclosure provides methods for generating a quality score, which facilitates, but is not necessary to perform the methods of the disclosure. For example, the methods of the disclosure may generate a large (or small) number of links, each of which may have a high quality score. In that case all the links may be used without filtering or otherwise evaluating the quality of the links. However, a link quality score has been found to be a useful measurement to distinguish true and false links. Accordingly, FIG. 3 provides a flow diagram of a method 300 for generating a link quality score by calculating a spatial null distribution 310 and a genomic null distribution 320. At step 310, the method may proceed by calculating a genomic empirical null distribution from a plurality of read pairs on a flow cell having approximately zero probability of being linked with one another. For example, read pairs that are on opposite surfaces of a flow cell cannot be truly linked with one another because the template molecule cannot flow across the space separating the flow cell surfaces. In another example, read pairs that are sufficiently distant from one another that fragments from an original DNA molecule could not have separated on a flow cell by that distance, could also be chosen to derive a null distribution because they also should not be linked. “Distant” in this context may refer to a predetermined threshold distance that would provide a high probability that read pairs are not from the same origin. A sufficient distance to conclude that two read pairs are not from the same origin can depend on the specific context and the underlying statistical distribution of the data. In general, a sufficient distance would be a distance that is significantly larger than the average separation between read pairs based on what would be expected under basic assumptions about the distribution of fragmented DNA sequences on the surface of the flow cell can indicate that the two objects are not from the same origin. Accordingly, the identification of links that are too distant on the flow cell surface to have originated from the same nucleic acid molecule helps in estimating the genomic null distribution of the entire dataset. These distant links, being non-candidate links due to their spatial separation, provide valuable data for understanding the background noise within the sequencing process. These estimates are readily gathered by selecting for clusters that are on opposite surfaces or are labeled with spatial data that are at distant opposite ends of the flow cell (e.g., the beginning and end regions of the flow cell). Accordingly, this subset of the total sequencing data may be used generate an estimate of the genomic null distribution. In some embodiments, a full null distribution is not necessary to generate a meaningful link quality score.

In some embodiments, the read pairs used to derive null distributions (either spatial or genomic) have near zero probability of being truly linked to each other (i.e., originating from same molecule). As described above, cross-surface read pairs may be used to derive genomic distance null because selecting for these allows for the method to select read pairs that are not from the same molecule but without otherwise setting a constraint on the genomic distance. Accordingly, some embodiments may avoid a biased null distribution, where the bias may be introduced by arbitrarily selecting a large genomic distance that may introduce a non-representative sample of reads with biased read length, for example. Similarly, to calculate spatial distance null, the disclosed methods may identify a set of reads that are not from the same molecule without setting any constraints on the spatial distance between them so as not to bias my spatial null distribution. As described above, cross-chromosome reads may be used to derive a null distribution without biasing the population used for the estimation.

A genomic null distribution allows for a determination of the expected level of noise or random variation in data, so the genomic null distribution provides a basis for statistical testing. In one formulation, the probability (p-value) of observing a given genomic linkage may be calculated under the default expectation of no linkage. If the observed result is unlikely to occur by chance (for example, if a p-value is below a predetermined threshold), the result provides evidence for rejecting the default expectation and suggests a genuine genomic link.

The genomic null distribution may be compared with the observed data to identify significant genomic signals. For example, discrepancies between the observed data and the genomic/spatial distance null distribution would be due to the presence of true links. In some embodiments, a significant signal means that there is strong evidence to suggest that an effect exists in the data that goes beyond random variability. This determination is often made using statistical tests that compare the observed data to a null distribution (random or expected distribution) and calculate a p-value. If the p-value is below a predetermined threshold (often denoted by a, such as 0.05), the result is considered significant. As described above, the genomic signals of interest would be genuine links between read pairs that connect read pairs on a flow cell that originated from the same polynucleotide sequence. Similarly, the quality of experimental procedures and data processing methods may be assessed by comparing the observed data with the genomic null distribution. Discrepancies between the observed data and the null distribution may indicate issues such as batch effects, sample contamination, or technical biases.

Calculating a genomic null distribution of read pairs on a flow cell, with an assumed zero probability of linkage, may be performed after aligning the reads to a reference genome. The alignment process maps the short DNA sequences (reads) obtained from the flow cell to their corresponding genomic locations in the reference genome. After alignment, reads may be processed to identify read pairs that align properly and are in the expected orientation. Additionally, after the alignment of read pairs to a reference genome, the process of assigning them to specific chromosomes may include analyzing the alignment information and determining the genomic location of each read pair. A mapping process may provide this information by indicating the mapped position of each read pair to the reference genome. For example, to assign read pairs to chromosomes, the mapped positions are examined against each chromosome in the reference genome that can be represented by a range of genomic coordinates. If the mapped position for a read pair falls within the range of a specific chromosome, the read pair is assigned to that chromosome. This assignment can be performed using a variety of methods, such as comparing the mapped positions to a pre-defined template chromosome coordinate file or utilizing a reference genome annotation that provides the chromosome information for each genomic position. Additionally, specific alignment tools or bioinformatics pipelines may provide built-in functionality for assigning reads to chromosomes based on the alignment results.

The method 300 may proceed to step 320, by calculating a spatial null distribution of a plurality of read pairs having approximately zero probability of being spatially linked with one another. Read pairs may be aligned to a reference genome where the read pair may be given a genomic location, for example, a genomic location on a chromosome. When two read pairs are on different chromosomes, those two read pairs are not likely to be linked because the reads came from different chromosomes in a reference genome (i.e., different molecules). Note that in some cases, there is a possibility that these reads might be involved in a structural variation (SV), such as a translocation or fusion event. In such cases, the reads, despite appearing on different chromosomes, could indeed be linked, thereby challenging the initial assumption of their independence. Challenging the null hypothesis with the consideration of structural variations (SVs) in sequencing data is not typically a significant factor for several reasons. Firstly, the occurrence of SVs in the genome is relatively rare. In the vast landscape of genomic sequences, the instances where read pairs from different chromosomes are linked due to SVs, such as translocations or fusions, are infrequent. As a result, the majority of read pairs on different chromosomes are indeed unlinked, originating from separate chromosomal molecules. This rarity means that the proportion of reads potentially linked by SVs is small when compared to the total number of reads, thus minimizing their impact on the overall data interpretation.

Additionally, by carefully selecting regions without a structural variant, the likelihood of encountering reads linked by SVs is reduced, which effectively reduces the potential influence of SVs on the null hypothesis. Another consideration is the statistical significance of SVs in large-scale genomic data. The sheer volume of unlinked reads in a typical sequencing project tends to dilute the impact of the relatively few reads linked by SVs. This statistical perspective ensures that the overall effect of SV-linked reads on the dataset is minimal, preserving the integrity of the data analysis. Accordingly, an estimate of the spatial null distribution may be generated by this subset of the total sequencing data which indicates read pairs for reads on different chromosome pairs.

Once a null distribution is calculated, the method may proceed to step 325, by calculating a false positive rate proportional to the product of the percentage of spatial and genomic null links below a threshold spatial and genomic distance. It should be realized that alternative methods for calculating a link quality score may not employ a false positive rate as recited in step 325, and thus it may be optional. For example, other positive and negative predictive values may be used to test true positive and true negative results. Link quality scores can be derived from a null distribution to assess the reliability or confidence of the observed data. These scores can be based on metrics such as the signal-to-noise ratio, the deviation from the null distribution, or the likelihood of observing the data given the default expectation. Higher link quality scores indicate greater reliability and confidence in the observed results. A statistical test of interest may be performed on the observed data to obtain test statistics or p-values for each expectation (false positive, false negative, true positive, true negative), The test statistics may be calculated on sampled data, where the p-values represent the probability of obtaining test statistics as extreme as, or more extreme than, the observed values under the default expectation. In other examples, a false positive rate may be used as an intermediate metric to estimate a false discovery rate, or any other type of type I error metric.

The method may then proceed to step 330 by generating a link quality score representing a probability of incorrectly assigning a link between the two polynucleotide reads. The link quality score may be generated based on the calculated genomic null distribution and the calculated spatial null distribution. Calculating the false discovery rate (FDR) from a null distribution may involve determining the proportion of false positives among the total positive findings when no true effects exist. A null distribution may be generated by simulating data under the assumption of no true effects or associations. The disclosure provides methods that may avoid steps of fitting a null distribution or simulating a null distribution by random shuffling of the observed data. As an alternative, the disclosure provides methods that take advantage of complementary information on the genomic sequences (spatial linking data) and may select a subset of that data, which would not present realistic or likely scenarios, to estimate a null distribution and generate a link quality score. Accordingly, systems and methods of the disclosure may advantageously increase the quality and efficiency of data processing associated with polynucleotide sequencing. In particular, practical applications of quantifiable linked reads may include improvements in mapping, and variant calling.

At step 335 the method calculates link quality scores for different combinations of thresholds. In some embodiments, the step 335 may include calculating a link quality score by determining null distributions for each threshold. In some embodiments, step 335 may include calculating a link quality score by determining a false discovery rate for each threshold. In some embodiments, step 335 may include calculating a link quality score by determining a prior null distribution that is used for a new threshold. In some embodiments, step 335 may include generating a link quality score based on a false discovery rate. By utilizing multiple or different combinations of thresholds, different link quality scores can be achieved, thereby allowing for more informed decisions or more accurate placement of linked read pairs. To calculate link quality scores for different combinations of thresholds, various parameters and metrics may be considered such as, the number of linked read pairs, the sequence depth, read overlap, sequence quality, and alignment scores. In some embodiments, an alignment score may be the output from a Smith-Waterman alignment. For each combination of these metrics, a threshold may be established, above or below which a link is considered to meet that particular quality criterion. Then, a link quality score can be calculated based on how many thresholds are met and how well they are met. This may be achieved through weighted averaging, or a composite linking quality score may be generated. This composite linking quality score may be designed to represent the individual scores into a comprehensive metric or series of metrics that encapsulate the overall quality of the links in a sequencing experiment. As an alternative or in addition, the outputs from different thresholds can be used in a hierarchical manner. High-quality connections, as determined by applying the most stringent thresholds, may be used, or analyzed, first. This may be beneficial in applications where computational resources are limited or where there is some benefit in identifying the most reliable links quickly. Lower-quality connections, which meet less stringent thresholds, may be considered later or as supplementary to the high-quality links.

Once the link quality scores have been calculated at the step 335, the process 300 can move to a decision step 340 to determine if a target, or optimal, threshold has been reached. If the target threshold has been reached, then the process 300 can terminate at an end step. If the target threshold has not been reached, then process 300 may return to step 325 so the calculations can be repeated in order to determine a target, or optimal threshold. A predetermined target threshold for identifying genuine connections between read pairs may be set based on prior knowledge from a prior validation. For example, the target threshold might be based on setting a minimum number of linked read pairs or a maximum allowable distance between linked read pairs on a flow cell. An optimal threshold may be determined by post-analysis of previous target thresholds. A threshold may be tailored to maximize specific metrics that may be important for the given task, such as sensitivity, specificity, or a balanced combination of both. Methods such as cross-validation may be used to fine-tune this target threshold in order to minimize errors, including false-positive and false-negative linked connections between read pairs.

Optionally, at step 350, various assay parameters may be tuned, including the nonlimiting examples of flow cell design, coverage, DNA loading density, DNA loading method, DNA loading strategy, the intensity of an electric field applied to a flow cell, linearity of the flow on the flow cell, the 3D structure of the DNA, a solution applied to the flow cell, and indexing. Flow cell design includes pitch size, well depth, and flow cell size.

In some embodiments, generating a link quality score includes generating a false positive rate. Note that there are several statistical tests that may be used to generate a link quality score that may or may not be directly associated with a false positive rate or false discovery rate. In general, the false positive rate is proportional to the product of the percentage of genomic null links and the percentage of spatial null links. Accordingly, calculating a link quality score may include generating an expected percentage of genomic-null links and spatial-null links for links from the plurality of read pairs on the flow cell below the threshold levels for spatial distance and genomic distance. The resulting expected percentage of genomic-null links and spatial-null links will also be proportional to the product of the false positive rate and the number of links from the plurality of read pairs on the flow cell below the threshold levels for spatial distance and genomic distance. In some embodiments, generating a link quality score includes generating a false discovery rate. A false discovery rate may be proportional to an estimated number of false links divided by the total number of links in the plurality of read pairs on the flow cell. The false discovery rate may be represented using the Phred scale.

In some embodiments, the link quality score includes a score representing the likelihood that a read from a particular cluster is linked to another cluster that originated from the same original target polynucleotide. In some embodiments, the link quality score of read pairs from two clusters is 0. In some embodiments, the link quality score of read pairs from two clusters is above 30. In some embodiments, location information includes a first spatial coordinate and a second spatial coordinate in a 2D coordinate system. In some embodiments, the method further includes sorting the plurality reads determined from the nucleic acid clusters by their spatial coordinates. This link quality score may be analogous to a mapping quality (MAPQ) score, e.g., likelihood that the mapping location of a read to a particular target polynucleotide is incorrect. The link quality score provides a new complementary type of information. For example, the linking quality score reflects proximity information that is distinct from other technologies aimed at contiguity or origin information. Proximity information informs how close read pairs are to each other within some threshold but does not require the read pairs to be directly adjacent (or contiguous with linked reads). Proximity information for polynucleotide fragments bound to a flow cell refers to the spatial relationship and relative positioning of these fragments on the surface of the flow cell during sequencing. Accordingly, the methods are adapted for this complementary information and in general, more linking information may be determined at a lower cost relative to other methods.

In some embodiments, the link quality score may be used in combination with a mapping quality (MAPQ) score to generate more high-quality mapping and to assign the nucleic acid sequence reads to target nucleotides. For example, a first read pair with low quality MAPQ score may be linked to a second read pair with a high quality MAPQ score, such that the first read pair can be aligned with high confidence. The linking quality score provides a metric to quantify the high confidence.

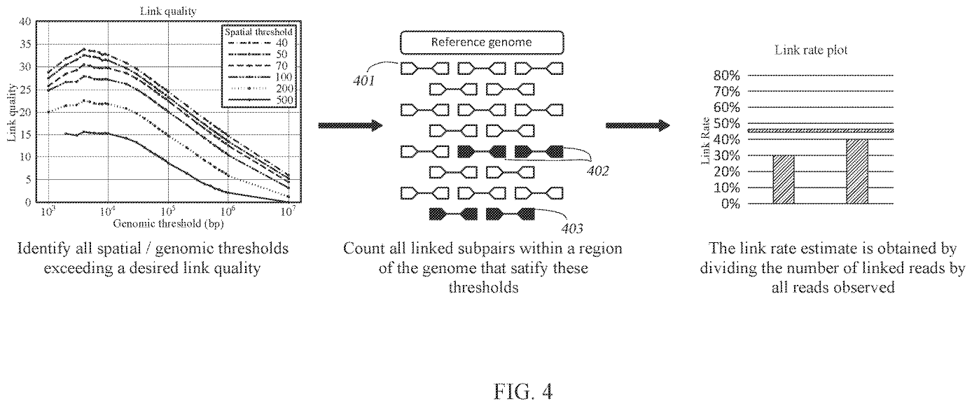

FIG. 4 is composed of three distinct panels, showing an overview of a method for estimating the percentage link rate. The link rate estimate may be obtained by dividing the number of linked reads by all reads observed and is the proportion of observed links (or connections) between sequence reads, relative to the total sequence reads. The link quality score is useful for quantifying improvements to the link rate and subsequent coverage. By evaluating link quality across thresholds and comparing results from different experiments, researchers can make informed decisions about the relevance and accuracy of their genomic studies.

The figure is divided into panels A, B, and C. The first panel A, labeled “Link quality,” exhibits a set of curves, each representing different spatial thresholds (40, 50, 70, 100, 300, and 500). The x-axis of this graph is the genomic threshold, spanning from 10\hat{ }3 to 10\hat{ }7 base pairs (bp). The y-axis denotes the link quality, which ranges from 0 to 40. The curves show how link quality changes across various genomic thresholds for the specified spatial thresholds. As the genomic threshold increases, the link quality seems to first increase slightly and then decrease, forming a downward trend. The region in the graph with a light green background emphasizes the area where the link quality is highest (and desirable for later applications) for various spatial thresholds.

The second panel, titled “Reference genome,” displays an array of shaded shapes (402, 403) each representing read sequences that cover the reference genome with a depth represented by the vertical stacking of the read sequences. Some of the sequence reads are shaded the same color (402), which indicates that those reads have linked read pairs. The panel also shows the unlinked read pairs (401) in light grey, which would be expected to form a percentage of the total number of sequence reads. This panel serves as a visual representation of the genomic regions under consideration in the analysis.

The third panel presents a histogram demonstrating link rates from two distinct hypothetical experiments. The y-axis represents the link rate, with percentages from 0% to 80%. The histogram provides two orange-colored bars of varying heights. The difference in their heights indicates the variance in link rates between the two experiments and/or two different conditions or treatments. The histogram serves as an example tool to monitor and adjust sequencing experiments, sample preparation, and methodologies to optimize the balance between linked reads and overall coverage to achieve, for example, a maximum linked read coverage. Variables that contribute significantly to enhancing the number of successfully linked reads can be identified by comparing the link rates between different conditions. For instance, in scenarios where a sample is scarce or the DNA quality is compromised, adjustments can be made to the sequencing protocol, such as altering the DNA input, the library preparation method, or the sequencing procedures and additives to improve the linkage quality and achieve the desired outcome. The ability to make informed decisions about these trade-offs via a linking quality score can enhance the efficiency of the experiment and the quality of the sequencing data. This flexibility and adaptability underscore the innovative aspect of the technology, providing a way to dynamically monitor and refine sequencing efforts.

Various assay parameters may be tuned, including the nonlimiting examples of flow cell design, coverage, DNA loading density, DNA loading method, DNA loading strategy, the intensity of an electric field applied to a flow cell, linearity of the flow on the flow cell, the 3D structure of the DNA, a solution applied to the flow cell, and indexing. Flow cell design includes pitch size, well depth, and flow cell size.

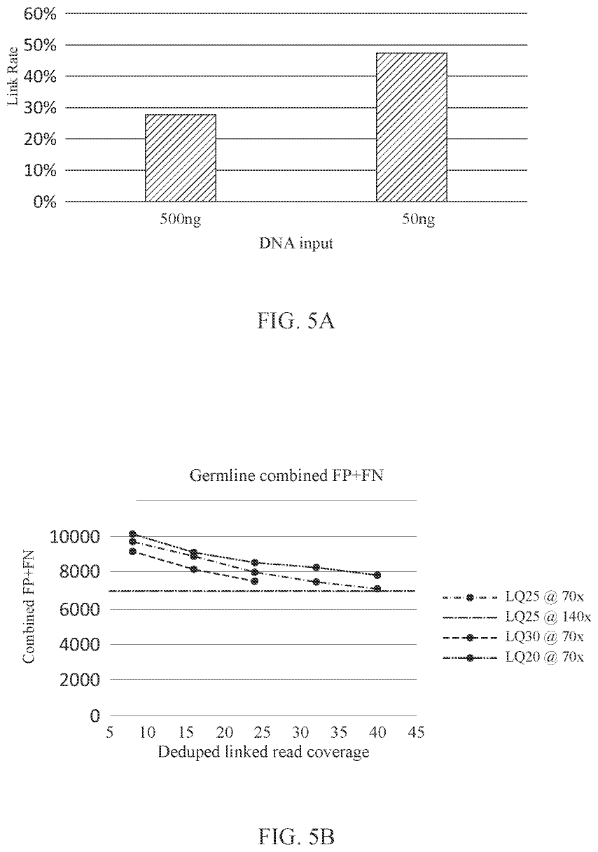

FIG. 5A shows a bar chart demonstrating the difference in link rates for one example of processing conditions on a sequencing system and for two absolute amounts (or concentrations) of DNA input each. In the experiment, an extraction of genomic DNA was performed, typically yielding DNA fragments greater than 60 kb in size, for sequencing at loading inputs of 500 ng and 50 ng. The different inputs lead to differences in the link rates. The observed variations in link rates suggest that the quantity of DNA loaded onto the flow cell can have a notable effect on the success of linkage across the reads. This relationship between loading input and link rate is an important consideration for optimizing sequencing experiments. Higher DNA inputs may enhance the chances of reads being linked, due to increased opportunities for overlapping reads; however, this must be balanced against the potential for overcrowding the flow cell, which could adversely affect the quality of sequencing. These differences can be further tuned and would be expected due to differences in the fluidics of between systems, from different recipes and flow cell geometries. The variability in link rates can also be attributed to the differences in the sequencing systems themselves, including the fluid dynamics and the physical design of the flow cells. These systems can vary in their handling of samples and reagents, and in the distribution patterns of DNA fragments during sequencing. Different sequencing systems may require individualized optimization of DNA input amounts to achieve the efficient linkage and coverage. Understanding these nuances via a linking quality score allows for fine-tuning of experiments to suit the potentially unique characteristics of sequencing systems.

FIG. 5B shows a line graph depicting the relationship between deduped linked read coverage and the combined false positive (FP) plus false negative (FN) rates for germline detection. There are three lines, each representing a different link quality score threshold: Q25 at 70× coverage, Q25 at 140× coverage, and Q30 at 70× coverage. The x-axis, which represents deduped linked read coverage, ranges from 0 to 45. The y-axis represents the combined FP+FN and ranges from 0 to 12000. The lines on the graph show that as deduped linked read coverage increases, the combined FP+FN decreases, which suggests an improvement in accuracy. However, the rate of improvement in accuracy seems to slow down as coverage exceeds approximately 30×, indicating diminishing returns with further increases in coverage. Each line on the graph flattens as it moves right, reinforcing the idea of a plateau effect in performance gains. The line representing Q25 at 70× appears to have the highest FP+FN rates across all coverage levels compared to the other two lines, while the Q30 at 70× generally has the lowest. The Q25 at 140× line shows intermediate performance between the Q25 and Q30 at 70× lines.

Flushing during the loading of the sample is expected and observed to lead to higher linked coverage. Consider loading a sample with a low concentration, and a single flush to remove excess reagent and unbound sample. This single flush at a low concentration would be expected to lead to a reduced occupancy of binding locations of the flow cell, but also a low false positive rate as described above, due to the lower chance of crowding leading to spurious links. This single flush using a relatively lower concentration is anticipated to result in fewer occupied binding sites on the flow cell. However, it also contributes to a lower false positive rate, as previously mentioned, due to the decreased likelihood of crowding that can cause spurious links. Consequently, this lower false positive rate is conducive to achieving both higher link quality and higher link rates. It's important to note that the term “lower concentration” is relative to the initial concentration used; if this lower concentration represents the optimal level, then the overall linked coverage for a single experiment may not necessarily be low, but could instead be at an optimal level that balances link quality and coverage. In contrast, if a sample is prepared with a single flush at a higher concentration, there may be increased occupancy and increased false positives, leading to a reduced link rate and also a suboptimal linked coverage.

Accordingly, one method of increasing the link rate is to perform multiple flushes at low concentration. The first flush at a low concentration would be expected to lead to a reduced occupancy of binding locations of the flow cell, and a low false positive rate. A second flush at a low concentration would then occupy the binding locations not occupied during the first flush. Fragmentation may be optionally performed between the flushing steps. Accordingly, after a second flush, the fragments from the second flush are not overly mixed with the bound fragments of the first flush. The flush may be performed two, three, four or more times. This methods leads to increased occupancy of the flow cell that leads to overall more sequences. The low false positive rate leads to higher link rates and in turn an overall increased linked coverage for a sequencing run. In some embodiments, single flush might yield a higher link rate. Despite this, retaining the multiple flush method as an option remains viable, particularly when tagmentation is employed between flushes. This technique has been observed in NSX runs to maintain high link rates even with multiple flushes, suggesting that the effectiveness of this strategy may vary.

### Improved Linking Rate Methods

Methods according to the disclosure may be adapted to different sequencers thereby increasing the link rate and leading to an overall increased linked coverage for a sequencing run. The amount of improvement in link rate for each system may be different depending on, for example, the density of nanowells on the flow cell or the flushing steps during the sequencing experiment. Accordingly, the disclosure provides for methods that achieve at least a threshold performance of increased linking rate. Additionally, the disclosure provides for methods of sequencing after the linking rate has been optimized, which does not require repeated optimization steps during the sequencing process. In some embodiments, methods may achieve a relative performance gain versus normal operation methods, such as methods performed without determining spatial information of reads on the flow cell surface.

In some embodiments, improved methods may be quantified based on the linking rates between linked sequence reads that have a high mapping quality score. In some embodiments, the linked sequence reads have at least a mapping quality score (MAPQ) of 25. In some embodiments the linked sequence reads have a MAPQ of any of at least 15, 16, 17, 18, 19, 20, 21, 22, 23, 24, 25, 26, 27, 28, 29, 30, 31, 32, 33, 34, and 35. In any of these embodiments, the linked sequence coverage may be 30× coverage. In some embodiments the linked sequence coverage may be any of at least 10×, 15×, 20×, 25×, 35×, 40×, 45×, 50×, 60×, and 70×. In some embodiments, the linked sequence reads have at least a mapping quality score (MAPQ) of 25, and the linked sequence coverage is an average of at least 30× coverage of linked sequence reads.

In some embodiments, improved methods may be quantified based on the link quality score. As described herein, the linking quality score may be quantified based on the phred scale. Accordingly, in some embodiments, the linked sequence reads have at least a link quality score of 25. In some embodiments the linked sequence reads have a link quality score of any of at least 15, 16, 17, 18, 19, 20, 21, 22, 23, 24, 25, 26, 27, 28, 29, 30, 31, 32, 33, 34, and 35. In any of these embodiments, the linked sequence coverage may be 30× coverage. In some embodiments the linked sequence coverage may be any of at least 10×, 15×, 20×, 25×, 35×, 40×, 45×, 50×, 60×, and 70×. In some embodiments, the linked sequence reads have a link quality score of 25, and the linked sequence coverage is an average of at least 30× coverage of linked sequence reads.

In some embodiments, improved methods may be quantified based on the percentage of sequence reads on the flow cell with a non-spurious link to at least one other sequence read. In some embodiments, the percentage of sequence reads on the flow cell with a non-spurious link is greater than 45%. In some embodiments, the percentage of sequence reads on the flow cell with a non-spurious link is greater than 45% and the average sequence coverage is at least 70× coverage for all sequence reads. As described above, the percentage of links with non-spurious links may increase by underloading the sample leading to lower overall coverage. Accordingly, the methods according to the disclosure may be quantified based on linking rate and on coverage. In some embodiments, the coverage may be coverage of linked sequence reads. In some embodiments, the coverage may be coverage of all sequence reads. In some embodiments, the percentage of sequence reads on the flow cell with a non-spurious link to at least one other sequence read is greater than 35% and the average sequence coverage is at least 90× coverage for all sequence reads. In some embodiments, the percentage of sequence reads on the flow cell with a non-spurious link to at least one other sequence read is greater than 25% and the average sequence coverage is at least 120× coverage for all sequence reads.

### Error Types Useful for the Link Quality Score

The disclosure provides for methods that determine a confidence level for linked data. In some embodiments, a confidence level for linked data may be related to the probability that a false link is present in a set of links that are filtered by a threshold level. The probability that a false link is in a set of links is type I error, also known as a false positive error. Type I error occurs when a default expectation that is actually true (that there is no link) is incorrectly rejected in favor of an alternative expectation (that there is a link). Several metrics may be used to assess and quantify Type I error rates, including the false positive rate (FPR) and the false discovery rate (FDR). Various metrics may be used to determine a confidence level for linked data.

For example, the false positive rate (FPR) represents the proportion of negative instances or cases where the default expectation is true but is incorrectly rejected as significant. FPR measures the rate at which an experiment incorrectly identifies a non-existent effect. FPR can be calculated as the ratio of false positives to the total number of true negatives plus false positives. Accordingly, the false positive rate (FPR) may be calculated using the following equation: FPR=FP/(FP+TN). Here FP represents the number of false positives (instances incorrectly classified as positive), and TN represents the number of true negatives (instances correctly classified as negative).

The above equation expresses the proportion of instances that were actually negative but were classified as positive by an expectation test (or binary classification model). The false positive rate may be used to assess the performance of classification models or the error rate in statistical expectation testing, indicating how often the test or model makes a Type I error (incorrectly rejecting a true default expectation).

The false discovery rate (FDR), is a related but distinct metric used primarily in multiple expectation testing scenarios. FDR may be used to measure the proportion of positive results (significant findings) that are false positives among all significant results. In other words, FDR addresses the issue of the proportion of incorrect rejections among all rejections. FDR may be used with multiple statistical tests conducted simultaneously. FDR may be controlled using various methods, such as the Benjamini-Hochberg procedure, for example, to limit the number of false discoveries. The FDR addresses the challenge that arises when conducting numerous statistical tests simultaneously, increasing the likelihood of obtaining statistically significant results by chance alone.

The False Discovery Rate (FDR) reflects the proportion of outcomes identified as significant that are, in fact, false positives. The false discovery rate (FDR) can be calculated using the following equation: FDR=FP/(FP+TP). Here, (FP+TP) represents the total number of data points, where FP represents the number of false positives (instances incorrectly classified as positive), and TP represents the number of true positives (instances correctly classified as positive).

This equation quantifies the proportion of instances that were classified as positive but are actually negative in binary classification problems. The false discovery rate may be used to control the proportion of erroneous positive findings among all the results deemed significant in multiple expectation testing. By quantifying the proportion of false positives within a selected population the FDR may balance between the number of true discoveries and the risk of making false positive declarations.

In some embodiments, the FDR offers a practical way to strike a balance between identifying meaningful associations in population health research and minimizing the risk of erroneous claims. By determining the FDR for a pre-defined threshold, the value of an FDR may be used to differentiate between genuinely meaningful associations and spurious findings. In some embodiments, the FDR may be tuned using FDR-controlling procedures, which are designed to control the FDR, which is the expected proportion of “discoveries” (rejected null hypotheses) that are false (incorrect rejections of the null). Equivalently, the FDR is the expected ratio of the number of false positive classifications (false discoveries) to the total number of positive classifications (rejections of the null). The total number of rejections of the null include both the number of false positives (FP) and true positives (TP). FDR-controlling procedures provide less stringent control of Type I errors compared to family-wise error rate (FWER) controlling procedures (such as the Bonferroni correction), which control the probability of at least one Type I error.

Some embodiments may also relate to an estimation of the number of true links that are not being used because they are above threshold levels. Type II metrics may be used to determine whether there more data that could be rescued. Type II error metrics assess the performance of statistical tests where they fail to detect true effects, i.e., they erroneously accept null hypotheses. Metrics such as False Negative Rate (FNR) and Sensitivity quantify the proportion of actual positives that are incorrectly classified as negatives or correctly identified as positives, respectively. Power and 1−β represent the probability of correctly rejecting a false default expectation, indicating the model's capacity to detect true effects. Miss Rate highlights the proportion of actual positive instances missed by the model. Specificity evaluates the accuracy of the model in correctly identifying negative instances, while Fall-out assesses its propensity to incorrectly classify negatives as positives. Type II Error Rate (β) expresses the probability of failing to reject a false default expectation when it is, in fact, false. These metrics collectively provide a comprehensive view of a model's or test's ability to avoid Type II errors and accurately identify true effects or outcomes.

In some embodiments, the type II metric may preferably be adapted to the size or scale that is commonly used or may be provided in a scale that provides intuitive meaning. For example, in genomics, the Phred-scaled quality scores have become a standardized way to assess the accuracy of DNA sequencing data, aiding researchers in making informed decisions about the reliability of identified genetic variations. This scale's logarithmic properties contribute to efficient data representation, sensitivity enhancement, and cross-platform comparability.

When a metric is subjected to the Phred scale, such as quality scores in sequencing data, its logarithmic nature helps amplify differences between values. Smaller differences in raw data may lead to larger differences on the Phred scale. This amplification of variations can aid in identifying subtle distinctions or anomalies that might be overlooked when examining raw data directly. Furthermore, the Phred scale's logarithmic transformation can facilitate comparisons across different experiments, instruments, or labs. Since the scale is standardized and logarithmic, it reduces the impact of variations between platforms or processes, making it easier to compare results consistently. The size of the values of false positive rates, can lead to a more favorable/interpretable quality metric. In some embodiments, FDR may be more preferable than FPR. In some embodiments, Type I error metrics may be preferable to type II error metrics. (More preferably FDR, instead of FPR).

To calculate an FDR, the observed test statistics or p-values would be compared with the null distribution. The proportion of positive findings above a specified threshold from the null distribution represents the expected number of false positives. Here, FPR is the proportion of false positives over all the pairwise comparisons being tested (so false links over the size of the null). An FDR is then calculated by dividing the number of false positives by the total number of positive findings (both true positives and false positives) obtained from the observed data. By comparing the observed test statistics or p-values with the null distribution, the FDR provides an estimate of the proportion of false positives among the significant findings. This quantifies the rate at which one would expect false positives to occur when the default expectation is true.

In some embodiments, linking the nucleic acid sequence reads includes determining the distance between each of the clusters and using the determined distance to assign reads to a specific target polynucleotide. In some embodiments, assigning the nucleic acid sequence reads includes determining for a link quality score that sequence reads from at least a first and a second cluster on the substrate derive from the same target polynucleotide. In some embodiments, sequence reads from more than two clusters are determined to be derived from the same target polynucleotide based on the link quality score. In some embodiments, the method further includes increasing the link quality score for sequence reads from at least the first cluster when the spatial distance and a genomic distance between the reads derived from first and second clusters are below a threshold value. In some embodiments, assigning the nucleic acid sequence reads to a particular target polynucleotide includes determining for a link quality score that a plurality of sequence reads from clusters on the substrate derive from the same target polynucleotide. In some embodiments, the method further includes increasing the link quality score for sequence reads from one cluster when the spatial distance and a genomic distance between one cluster and one or more other clusters are below a threshold value.

Linking reads can be performed in a genomic reference dependent or independent manner. For genomic independent linking, links are formed between reads using spatial information only. In a non-limiting example, reads are linked when they are 50 flow cell distance units apart. In this example, reference-based information (including, but not limited to, genomic information and the like) is not considered in linking these reads. For a genomic dependent linking, links are formed between reads using spatial and genomic information. In a non-limiting example of a genomic dependent linking, a pair of reads or multiple reads are linked when they are 50 flow cell distance units apart and within 10 kbp distance when the reads are aligned to a reference genome.

In some embodiments, the alignment data can be analyzed to create a genomic null distribution by considering the expected genomic properties of the read pairs. For example, if the reads are randomly sampled from the genome without any specific linkage, the distribution of distances between the mapped pairs should follow a random distribution pattern. In some embodiments, a genomic null distribution can be generated by calculating the observed distance distribution of properly aligned read pairs and comparing it to the expected distribution under the assumption of no genomic linkage. This can be achieved by various statistical methods. For instance, one approach may involve calculating the empirical distribution of distances between properly aligned read pairs and comparing it to a theoretical null distribution. The null distribution can be generated by randomly permuting the read pairs' mapping positions while maintaining their original genomic positions. By repeating this permutation process multiple times and generating null distributions, a statistical baseline may be established against which the observed distance distribution can be compared.

In some embodiments, calculating the genomic null distribution of the plurality of read pairs includes determining read pairs from clusters on the flow cell. In some embodiments, clusters on the flow cell may be in random flow cells or in patterned flow cells. In some embodiments, the location data of a read pair may be the location of a cluster on the flow cell. In some embodiments, a step of determining read pairs from clusters on the flow cell may be performed before, during or after the data analysis of the null distribution. There may be multiple labels for clusters of read pairs that may be used for calculating a null distribution. For example, clusters used for the null distribution may be spatially distant from one another on the same or opposite surfaces of a flow cell. In some embodiments, the clusters may be on the same surface but opposite ends of the flow cell. In some embodiments, the clusters may be opposite surfaces of the flow cell.

In some embodiments, the spatial null distribution of the plurality of read pairs may be calculated by determining read pairs from clusters on the flow cell. In some embodiments, calculating the null distribution includes estimating the null distribution based on a subsample of the total data produced in a sequencing run. In some embodiments, calculating a null distribution may be performed during the first few cycles of a sequencing run and used to determine if the experiment should continue. In some cases, a null distribution may be re-used for subsequent experiments.

In some embodiments, clusters may be used to calculate a null distribution where a first cluster has a read pair derived from a first chromosome and a second cluster has a read pair from a second, different chromosome. In some embodiments, calculating a null distribution may include applying threshold levels for spatial distance and genomic distance to this plurality of read pairs on the flow cell. In some embodiments, methods may include determining at least one threshold level such that read pairs from clusters derive from the same DNA molecule with a predetermined probability for spatial distance and genomic distance using the quality of the link. For example, methods may include estimating a linking rate for the total links between read pairs on the flow cell for the threshold levels. Accordingly, these methods may be used to select a threshold level based on the link quality score. Based on that quality level, different assay parameters can be explored and adjusted to evaluate the impact on the number and quality of links that are produced.

In some embodiments, the method includes calculating the genomic null distribution of the plurality of read pairs includes determining read pairs from clusters on the flow cell. In some embodiments, calculating the genomic null distribution of the plurality of read pairs includes determining read pairs from clusters that are spatially distant from one another on the same or opposite surfaces of a flow cell. In some embodiments, the clusters used for calculating the null distribution are on the same surface but opposite ends of the flow cell. In some embodiments, the clusters used for calculating the null distribution are opposite surfaces of the flow cell.

In some embodiments, the method includes calculating the spatial null distribution of the plurality of the plurality of read pairs includes determining read pairs from clusters on the flow cell. In some embodiments, the techniques described herein relate to a method, wherein a first cluster has nucleic acid derived from a first chromosome and a second cluster has nucleic acid from a second, different chromosome.

In some embodiments, a method may further include applying threshold levels for spatial distance and genomic distance to the plurality of read pairs on the flow cell.

In some embodiments, a method may further include determining at least one threshold level such that read pairs from clusters derive from the same DNA molecule with a predetermined probability for spatial distance and genomic distance using the quality of the link. In some embodiments, a method may further include estimating a linking rate for the total links between read pairs on the flow cell for the threshold levels.

In some embodiments, the method may further include selecting a threshold level based on the link quality score. In some embodiments, the method may further include adjusting an assay parameter based on the link quality score. In some embodiments, the assay parameter includes at least one of flow cell design, coverage, DNA loading density, DNA loading method, DNA loading strategy, the intensity of an electric field applied to a flow cell, linearity of the flow on the flow cell, the 3D structure of the DNA, a solution applied to the flow cell, and indexing. Similarly, in some embodiments, the assay parameter of flow cell design includes at least one of pitch size, well depth, and flow cell size.

In some embodiments, the techniques described herein relate to a method, wherein generating a link quality score includes generating a false positive rate. In some embodiments, the false positive rate is proportional to the product of the percentage of genomic null links and the percentage of spatial null links. In some embodiments, generating a link quality score includes generating an expected percentage of genomic-null links and spatial-null links for links from the plurality of read pairs on the flow cell below the threshold levels for spatial and genomic distance.

In some embodiments, the expected percentage of genomic-null links and spatial-null links are proportional to the product of the false positive rate and the number of links from the plurality of read pairs on the flow cell below the threshold levels for spatial distance and genomic distance. In some embodiments, generating a link quality score includes generating a false discovery rate. In some embodiments, the false discovery rate is proportional to an estimated number of false links divided by the total number of links in the plurality of read pairs on the flow cell. In some embodiments, the link quality score is represented using the Phred scale.

In some embodiments, the method relates to calculating a genomic null distribution and spatial null distribution of read pairs is performed on a subset of the plurality of read pairs on a flow cell. In some embodiments, the read pairs are representative of the total read pairs on the flow cell. In some embodiments, the quality of a link between the two polynucleotides represents the quality of the links for the plurality of reads on the flow cell below the threshold levels for spatial distance and genomic distance. In some embodiments, the quality of a link between the two polynucleotides is applied to individual read pairs within the plurality of reads on the flow cell.

In some embodiments, a method may further include assigning a candidate link between two polynucleotide reads using the spatial positional data. In some embodiments, assigning a candidate link between two polynucleotide reads using the spatial positional data includes determining a link quality score that a first cluster and a second cluster on sequencing flow cell originate from the same target polynucleotide. In some embodiments, generating a link quality score includes a statistical test including at least one of a t-test, a chi-square test, a permutation test, and a deconvolution analysis.

In some embodiments, the systems and methods may determine the appropriate threshold to remove read pairs that are nearby each other on the flow cell, but not derived from the same polynucleotide molecule. Different datasets may use different thresholds and the choice of threshold may relate to a trade-off between sensitivity (the ability to detect true links) and specificity (the ability to exclude false links). Setting a stringent threshold may increase specificity by removing more spurious links but can also result in the exclusion of genuine genomic associations. Conversely, setting a lenient threshold may increase sensitivity but can lead to the inclusion of false-positive links. Regardless, the confidence assigned to a link after establishing threshold is unclear without having a metric, such as the link quality score, to quantify the quality of the data after applying the threshold.

### Examples of Parameters Affecting Link Quality

The link quality scores associated with data obtained through the sequencing of a biological sample on a flow cell may be subject to the influence of various factors. Such factors include the precision of base calling, the selection of sequencing chemistry, the extent of read length, and the intensity of signals derived from fluorescent labels may all bear implications on the assignment of link quality scores. Additional considerations including the density of clustering on the flow cell, the cycle number at various stages of sequencing, the quality of the sample material being sequenced (such as the molecular weight of the polynucleotide), and the specific GC content of sequences can also contribute to fluctuations in link quality scores.

In some embodiments, methods according to the disclosure may use a concentration of less than 5 picomolar in a loading sample on the flow cell. In some embodiments, methods according to the disclosure may use a concentration of less than any of 1, 2, 3, 4, 5, 6, 7, 8, 9, and 10 picomolar in a loading sample on the flow cell. In some embodiments, methods according to the disclosure may use a loading sample of less than 75 nanograms. In some embodiments, methods according to the disclosure may use a loading sample of any of less than 25, 50, 75 and 100 nanograms. In some embodiments, methods according to the disclosure may use a loading sample of any of less than 250, 500, 750 and 1000 nanograms.

In some embodiments, methods according to the disclosure may use gentle handling techniques. In some embodiments, gentle handling techniques may be quantified by procedures used during sample preparation. In some embodiments, methods according to the disclosure may use fewer than three purification steps, such as two, one or no purification steps, which are performed on a loading sample prior to sample loading. In some embodiments, the length of the polynucleotide, determined as part of the quality control (QC) process, may be used to quantify the effectiveness of the sample preparation procedures as described. In some embodiments, the length of the polynucleotide may be known based on the typical output of an assay design. In some embodiments, methods may use polynucleotides with at least 60,000 base pairs in a loading sample. In some embodiments, methods may use polynucleotides with any of at least 10, 20, 30, 40, 50, 60, 70 80, 90 kbp, and 100 kbp in a loading sample.

In some embodiments, methods according to the disclosure may use a flow rate of less than 250 μL/min for the flow of solution across the flow cell during sample loading. In some embodiments, the methods may use a flow rate that is reduced by any of 10%, 20%, 30%, 40%, 50%, 60%, 70%, 80% and 90%. In some embodiments, the flow rate may be maintained at less than 250 μL/min, with specific instances employing rates such as 50 μL/min, 100 μL/min, and other similar graduations. In some embodiments, methods according to the disclosure may use a flow rate of greater than 250 μL/min for the flow of solution across the flow cell during sample loading. In some embodiments, the methods may use a flow rate that is increased by any of 10%, 20%, 30%, 40%, 50%, 60%, 70%, 80% and 90%.

In some embodiments, methods according to the disclosure may use a crude lysate loading sample. Utilizing a crude lysate as the loading sample is one innovative approach mentioned in the disclosure to potentially increase the link rate in sequencing methods. This strategy involves using the lysate, which is a mixture of cell components obtained after cell lysis, directly in the sequencing process, bypassing the conventional DNA extraction and purification steps. The rationale behind this approach is that crude lysates typically contain high-molecular-weight DNA, which is more likely to maintain physical links between different parts of the genome. When these high-molecular-weight DNA fragments are used in sequencing, there is an increased likelihood of capturing longer stretches of the genome that are physically linked. This can lead to an enhanced link rate, as more extensive genomic regions can be sequenced in a single read or in closely associated reads. Furthermore, by skipping the DNA extraction process, which can sometimes shear DNA into smaller fragments, the integrity of these long DNA molecules is better preserved. This preservation is useful for maintaining the possibility of the physical link between reads that in turn leads to a high link rate. In some embodiments, obtaining sequence reads may include loading a crude lysate loading sample onto the flow cell, where the crude lysate sample has been lysed but not extracted. In some embodiments, the crude lysate may include an unpurified mixture obtained by lysing a biological sample through mechanical, chemical, or thermal means, thereby releasing cellular contents. In some embodiments, crude lysate may be used directly in the sequencing process without further purification.

In some embodiments, methods according to the disclosure may sequence the polynucleotides after two or more loading cycles on the flow cell. In some embodiments outlined in the disclosure, the methods propose an approach of sequencing polynucleotides after conducting two or more flush loading cycles on the flow cell. This technique involves repeatedly introducing the sample into the flow cell, followed by flushing it out, and then repeating the process multiple times. The rationale behind this method is that multiple flush loading cycles can potentially enhance the distribution and occupancy of the polynucleotides on the flow cell surface. Each flush cycle has the potential to deposit different sets of polynucleotide fragments onto the flow cell. By repeating this process, the method may maximize the coverage of the genomic regions being sequenced, while also potentially reducing the amount of sample loaded during each cycle. This increased coverage is crucial for ensuring comprehensive genomic analysis, particularly in studies where detecting low-abundance sequences is relevant.

According to the disclosure, various sequencing methods have been observed to achieve different linking rates, and these rates can be used as a metric for optimization. By measuring the linking rate, adjustments can be made to the sequencing protocol to optimize this rate, which could involve changing the density of clustering on the flow cell, the cycle number, the quality of the sample material in terms of molecular weight. In some embodiments, the percentage of sequence reads with a link to a second sequence read is greater than 45%. To reach such a high link rate, the sequencing methods should be tuned and optimized. Methods according to the disclosure consistently achieve a link rate that meets or exceeds this threshold.

In some embodiments, sample loading includes introducing one or more additives. In some embodiments, sample loading includes introducing one or more additives selected from the group consisting of: Magnesium, detergents, betaine, DMSO, chelating agents, reducing agents, and PCR enhancers and stabilizers. These additives may be selected based on their ability to enhance the link rate of the sequencing process, but are commonly used in typical sequencing methods in different combinations or concentrations.

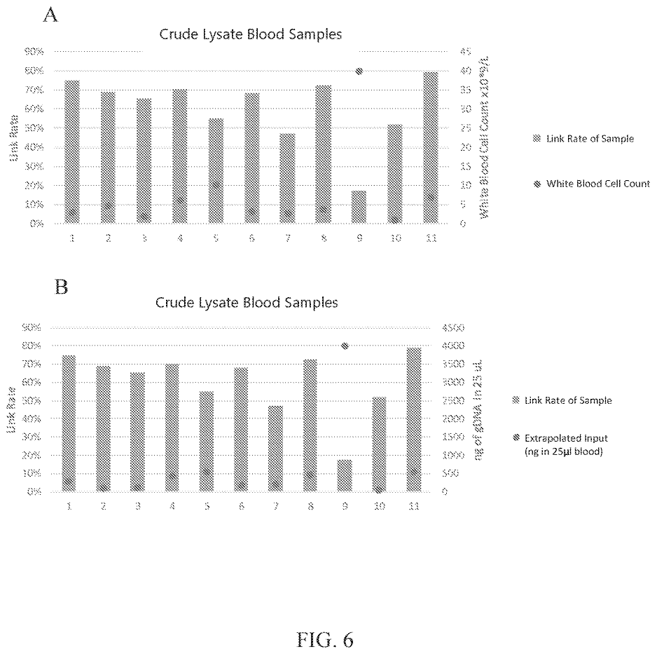

FIG. 6 features two panels of bar charts labeled “Crude Lysate Blood Samples” that report linked read rates for 11 patients, numbered 1 through 11. The chart displays two types of data: one is represented by orange bars and labeled “NS6K-Crude,” which corresponds to Q25 link rates for crude samples from crude lysate blood samples sequenced on a NovaSeq 6000s. These percentages align to the left vertical axis. In Panel A, the second type of data is represented by dots and labeled “WBC,” which stands for white blood cells, with counts shown on the right vertical axis. The bars show some variation in the link rate between patients, with percentages ranging from below 20% (for patient 9) to above 80%. The WBC counts are plotted on the same horizontal axis as the bars, with the highest count close to 40 (in thousands per μL) and the lowest count around 5. As observed in the right axis of the chart, patient 9 has an abnormally high white blood cell count, leading to a likely overloading of the sample on the flow cell and a corresponding reduction in the Q25 link rate. Panel B shows the same Link Rate data as Panel A, but the dots represent nanograms of genomic data found in 25 μl of blood.

The following experimental section details a nonlimiting example of procedures for handling various biological samples and extracting high molecular weight (HMW) DNA. Blood samples may be stored at 4 °C and processed within 3 days, using 25 μL per reaction and collected in K2EDTA. Bone Marrow Aspirate (BMA) samples have similar storage and processing requirements but use 15 μL per reaction and are also collected in K2EDTA. In some examples, a range of 15 μL to 30 μL may be used. Tissue culture cells, and/or fresh frozen tissue use harvesting through centrifugation or trypsinization, followed by cell counting. After harvesting, any liquid should be removed as much as possible, and the remaining cell pellets should be processed immediately or frozen at −80 °C.

For HMW DNA extraction, the following general considerations may assist in maintaining the integrity of the DNA: avoiding high temperatures, pH extremes, intercalating dyes, UV light, and vortexing. The DNA should be pipetted with wide bore tips, with all RNA removed, and without using guanidine thiocyanate or phenol/chloroform to minimize damage. The extracted DNA should then be resuspended in a low salt buffer or water. Several specific DNA extraction kits available from various suppliers are listed, where the general recommendation would be to follow the manufacturer's protocols for blood, bodily fluids, and tissue culture cells. If no specific protocol is available for tissue culture cells, prior experimental results would suggest using the same procedure as for blood.

For quality control (QC) and storage, DNA concentration should be checked on both Nanodrop and Qubit for concordance, and purity checked on Nanodrop with specified optimal optical density (OD) ratios. DNA size should be assessed using DNA genomic screentape or pulse field gel electrophoresis systems, aiming for a DNA Integrity Number (DIN) greater than 9. Finally, DNA should be stored at −20 °C for long-term storage, with a recommendation to avoid more than ten freeze-thaw cycles and to aliquot the DNA if necessary.

The following experimental procedure provides an example for methods of preparing blood and bone marrow aspirate (BMA) samples and involves several reagents and a two-step heat incubation process.

First, the liquid is removed from the cell pellets, which should be approximately 25 μL in volume. These pellets can be processed immediately or stored at −80 °C for later use. For the lysis step, a 10× lysis buffer containing 2% Sodium Dodecyl Sulfate (SDS), 500 mM Tris HCl pH 8.0, 10 mM Ethylenediaminetetraacetic acid (EDTA), and 5% Tween is used. This buffer is stored at room temperature. A thermolabile proteinase K solution, stored at −20 °C, and a 10× tagmentation buffer containing 50 mM Magnesium Acetate (MgAc) and 100 mM Tris Acetate (TrisAc) pH 7.6, stored at room temperature, are also utilized.

For the lysis mix, 25 μL of the lysis buffer, 30 μL of proteinase K, and 170 μL of water are mixed for blood or cell samples, while 180 μL of water is used for BMA samples. The sample volumes of 25 μL for blood or cells and 15 μL for BMA are then added to this lysis mix. This mixture is pipet-mixed with wide-bore tips and placed in a heat block set at 37 °C for 15 minutes, then transferred to a second heat block at 55 °C for 10 minutes. After the heat treatment, the samples are cooled to room temperature. Simultaneously, an inhibition mix may be prepared with 40% α-cyclodextrin and tagmentation buffer. Once the lysate has cooled, 50 μL of this inhibition mix is added to each sample, followed by pipet-mixing with wide-bore tips 10 times. The sample is then ready for loading onto a flow cell for further analysis.

As an alternative workflow, it is possible to use a different type of proteinase K, such as the one from Tritirachium album, which can be inactivated at a higher temperature of 75-80 °C for 10 minutes, or by using an inhibitor like Tetrapeptidyl chloromethyl ketone (TCK) at a concentration of 0.05-0.08 mg/mL, which can be combined with the inhibition mix.

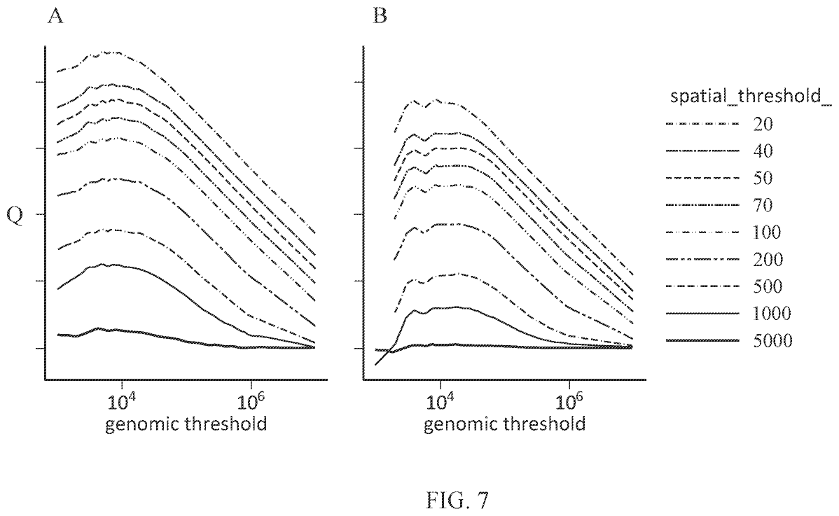

FIG. 7 shows two plots of calculated link quality scores for an experimental system by using estimated null distributions. The Y-axis represents the Q value, which is a logarithmic scale used to convey the accuracy, and where a higher Q value indicates a link of better quality. This value may be derived directly from the FDR. The X-axis represents the genomic threshold, while the different colors represent various spatial thresholds. As the genomic threshold increases, the link quality generally improves until reaching a peak. Beyond that point, increasing the genomic threshold further leads to a decline in link quality score. Each point on the plot corresponds to all the links created under a specific genomic threshold and spatial threshold. This plot enables the selection of a line, such as Q20, Q25, or Q30, and assign all the data points above that quality threshold with a corresponding link quality score. The highest link quality score threshold may be balanced with the number of links obtained from that threshold. This approach allows for quantifying desired thresholds or multiple thresholds based on a reproducible link quality score. Increasing the threshold, such as from Q20 to Q30, leads to a lower linking rate as selecting links becomes more conservative. If the threshold is too stringent, some percentage of reads with links may be eliminated. Accordingly, this method may be used to strike a balance between a desired Q score and the number of obtained links. Additionally, these thresholds may be used as direct inputs for downstream analyses, such as error correction or variant calling. Additionally, the method may iteratively eliminate lower linking rates which efficiently balances the sequencing system for downstream analysis. In this iterative process, one would start at a lower quality threshold, like Q20, which includes more links but potentially with lower confidence. As the threshold increases, for example to Q30, the criteria for link inclusion become more stringent, resulting in fewer but higher confidence links. This selective process eliminates lower quality links, leading to a compact dataset that is optimized for both link quality and quantity. By quantifying and setting desired thresholds, one can reproducibly achieve a balance that suits the specific needs of downstream analysis.

Similar variations can be observed in NovaSeq 6K runs. Certain runs exhibit lower linking quality compared to others. Factors such as the type of flow cell, experimental conditions (such as underloaded DNA or normal DNA amounts), distances between wells, well depth, and flow rate on the flow cell can all affect linking qualities. The distinction between the formation of links and the detection of links in genomic sequencing is useful to explain the usefulness of certain methods of the disclosure. The process of forming links is influenced by factors like the use of high molecular weight (HMW) DNA or pulsed loading, which affect how DNA fragments physically associate on the flow cell. On the other hand, setting a quality threshold is about determining our ability to detect these links. It's a measure of confidence in the links that we identify, rather than a measure of all the links that are present, including those that are undetectable. The utility of setting a quality threshold is to balance the number of reads that form detectable links. Each threshold, representing a different spatial distance, allows for the quantification of the quality of an experiment at a given threshold level. However, this approach quantifies only those links that are detectable, not all the links that are actually formed. In some embodiments, more links might occur but might not be detectable. Links that occur at a combination of genomic and flow cell distances that are indistinguishable from a null distribution rate would remain present but undetectable based on the assigned thresholds and methods of characterizing true and spurious links. For example, while improvements in linking rate might also affect a null distribution for unlinked reads, a sample with higher linking rate for reads that are very distant might be undetected because these distant links would exceed a threshold physical distance where a majority of the reads are unlinked. However, in general, a higher percentage, e.g., 40% or 50%, indicates better performance. This approach applies to NextSeq2K runs with higher loading of DNA as well as NextSeq2K runs with underloaded DNA. Finally, a composite of multiple thresholds, for example, combining all the thresholds results in a significantly improved linking rate.

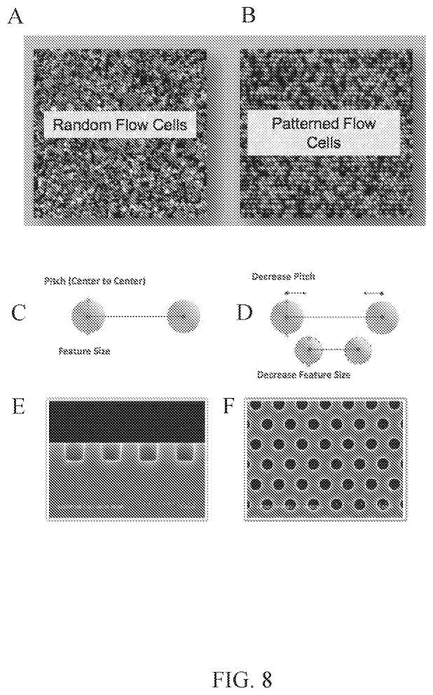

FIG. 8 shows a schematic of a patterned flow cell, and variable parameters of the patterned flow cell. As explained in FIG. 7, experimental conditions significantly impact the link quality scores obtained from the links in a particular run. FIG. 8 shows an example of a pitch and feature size of a patterned flow cell and a random flow cell. On the top, there are two images labeled respectively. The ‘Random Flow Cells’ image shows a multitude of colored spots scattered in a non-uniform distribution, representing a flow cell where the capture sites are randomly organized. The ‘Patterned Flow Cells’ image depicts a more ordered array of colored spots, illustrating a flow cell with systematically arranged capture sites. Below these images, there are diagrams explaining the concepts of ‘Pitch’ and ‘Feature Size’, along with the effects of ‘Decrease Pitch’ and ‘Decrease Feature Size’. ‘Pitch’ is defined as the center-to-center distance between adjacent features, represented by two orange circles with a line indicating the measurement between their centers. To the right, the concept of ‘Decrease Pitch’ is shown by moving the features closer together, as indicated by an arrow. Similarly, ‘Feature Size’ is depicted by the diameter of an orange circle, and ‘Decrease Feature Size’ is represented by a smaller circle with a dashed outline, suggesting a reduction in the size of the feature. At the bottom of the figure are two micrographs. On the left is a cross-sectional image of a flow cell with several wells or pits, to capture DNA molecules. On the right is a top-down micrograph of a flow cell surface showing a high density of uniform wells, which are the features where DNA molecules are captured during the sequencing process. The figure illustrates the structural differences between random and patterned flow cells and highlights the concepts of pitch and feature size in the design of flow cells, which are relevant parameters affecting methods of the disclosure. The comparison suggests that adjustments in pitch and feature size can influence the distribution and occupancy of DNA fragments on the flow cell, which in turn may affect the sequencing output.

The variable parameters of a flow cell, such as pitch and feature size may dictate several aspects of the sequencing process, including how densely the sequencing reads or fragments may be packed and how well the optical systems can distinguish between individual clusters. Changes in these variables may lead to differences in sequencing quality and/or the link quality scores between read pairs. For instance, a smaller pitch might allow for more DNA clusters but can also lead to overlapping signals between wells because of links originating from different proximal but unrelated wells, which would therefore include more of both true and false links, making it challenging to assign link quality scores to reads in the sequence. Conversely, a larger pitch might provide clearer resolution between true and false links, leading to a less significant false discovery rate, but at a cost of reducing the throughput or the number of sequences that can be read in parallel. A larger pitch might also increase the minimum size needed to span two nano wells with a single polynucleotide molecule. Experimental conditions including temperature, reagent quality, and flow rate, combined with design parameters illustrated in FIG. 8, may influence the link quality scores. For instance, poor experimental conditions may introduce noise into the data and change the biochemical process of fragmenting the biological sample, making it difficult to establish reliable links between reads pairs, thus leading to lower link quality scores.

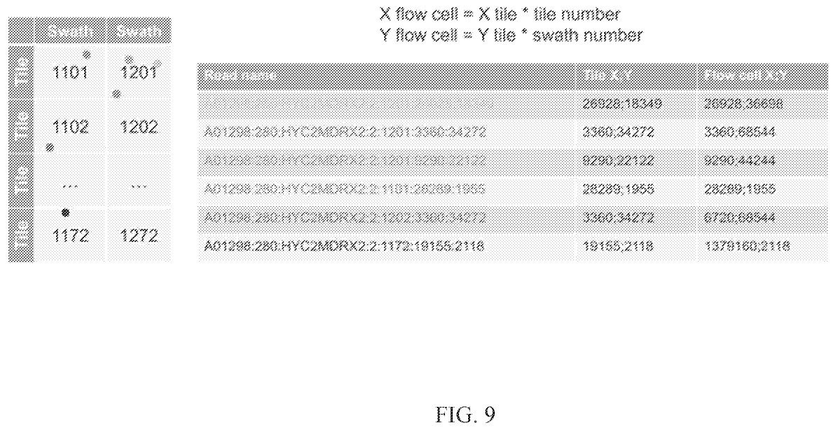

FIG. 9 shows how the Tile X-Y coordinates can be converted to flow cell X-Y coordinates, where the flow cell X-coordinate=Tile X-coordinate×Tile number; and the flow cell Y-coordinate=Tile Y-coordinate×Swath number. Flow cells commonly have different coordinate systems depending on the granularity of the data or the components of the system. In sequencing technologies, a flow cell is often divided into multiple tiles, each of which may contain numerous DNA clusters or spots. The Tile X-Y coordinates are a way of locating specific spots within this smaller unit (tile). FIG. 9 provides a formula for converting Tile X-Y coordinates to Flow cell X-Y coordinates. Specifically, the Tile X-coordinate may be multiplied by the Tile number to obtain the Flow cell X-coordinate. Similarly, the Tile Y-coordinate may be multiplied by the Swath number to obtain the Flow cell Y-coordinate. The Tile number indicates the specific tile of interest, while the Swath number may indicate a particular row or column of tiles on the flow cell. Multiplying the local coordinates by these values converts them into global coordinates that can be used to locate a spot anywhere on the flow cell. A similar process may be used to convert spatial coordinates to a unit of interest, such as a unit comparable to the length of a DNA molecule or the insert size.

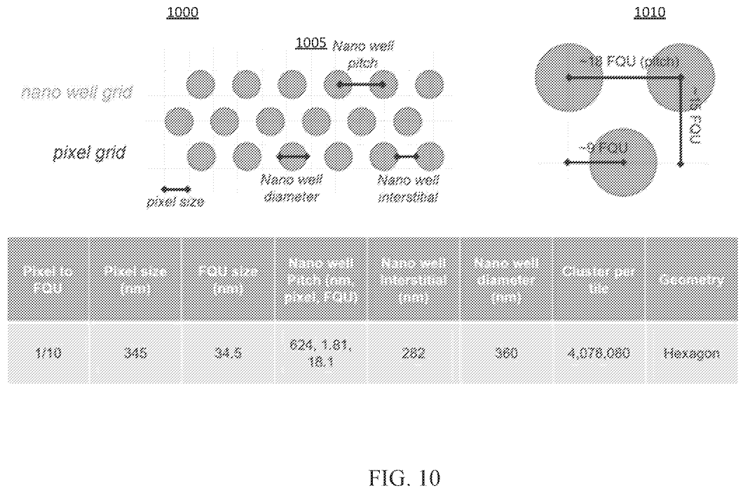

FIG. 10 provides a non-limiting example 1000 of flow cell grid geometry 1005 and spatial units 1010. As shown at grid 1005, the nanowells of the flow cell are organized into a grid. The nanowell grid 1005 is overlayed with a pixel grid (not shown) which is derived from an image of the flow cell grid 1005 by a CCD camera. In this example, 1 pixel in the captured image represents 10 FQU's and each pixel size is 345 nm. Various cameras at different magnification levels will have different pixel sizes and the pixel size disclosed in FIG. 10 is by way of example only. With the pixel size and the pixel to FQU ratio known, the physical FQU size is determined to be 34.5 nm, in this example. Other metrics are obtained with this information includes the nanowell pitch, diameter, and interstitial distance. In this example, the nanowell grid is organized in a hexagonal pattern.

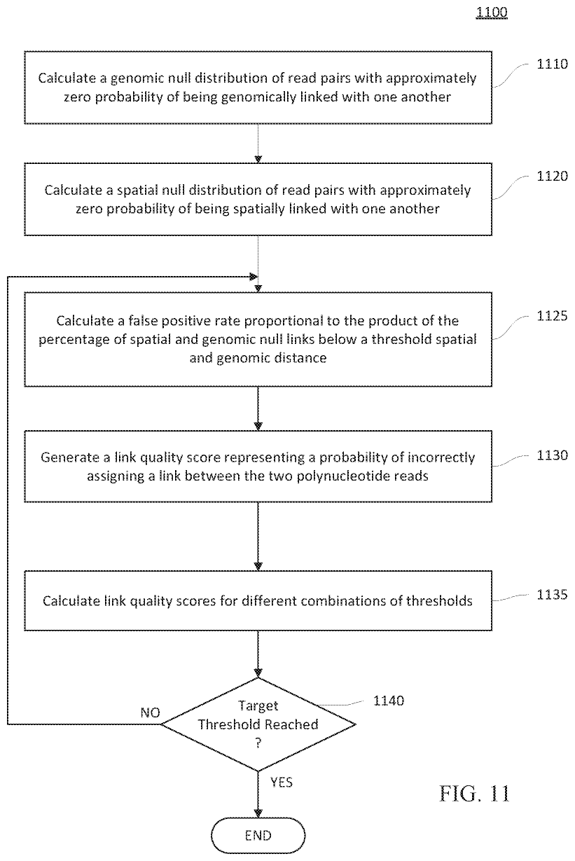

In one embodiment, FIG. 11 provides a flow diagram of a method 1100 for generating a link quality score by calculating a spatial null distribution and a genomic null distribution. At step 1110, the method may proceed by calculating a genomic null distribution of a plurality of read pairs on a flow cell having approximately zero probability. A genomic null distribution of read pairs can be estimated by selecting read pairs with approximately zero probability of being genuinely linked with one another. For example, read pairs that are on opposite surfaces of a flow cell cannot be truly linked with one another because the same template molecule cannot bind to both surfaces of the flow cell simultaneously as they appear on opposite sides of the flow cell. In another example, read pairs that are sufficiently distant from one another that fragments from an original DNA molecule could not have separated on a flow cell by that distance, could also be chosen to derive a null distribution because they also should not be linked. “Distant” in this context may refer to a predetermined threshold distance that would provide a high probability that read pairs are not from the same origin. A sufficient distance to conclude that two read pairs are not from the same origin can depend on the specific context and the underlying statistical distribution of the data. In general, a sufficient distance would be a distance that is significantly larger than the average separation between read pairs based on what would be expected under basic assumptions about the distribution of fragmented DNA sequences on the surface of the flow cell can indicate that the two objects are not from the same origin. Accordingly, identifying candidate links from links that physically cannot originate from the same nucleic acid molecule provides an estimate of the genomic null distribution of the entire data set. These estimates are readily gathered by selecting for clusters that are on opposite surfaces or are labeled with spatial data that are at distant opposite ends of the flow cell (e.g. the beginning and end regions of the flow cell). Accordingly, this subset of the total sequencing data may be used generate an estimate of the genomic null distribution. In some embodiments, a full null distribution is not necessary to generate a meaningful link quality score.

The method 1100 may proceed to step 1120, by calculating a spatial null distribution of a plurality of read pairs having approximately zero probability of being spatially linked with one another. Read pairs may be aligned to a reference genome where the read pair may be given a genomic location, for example, a genomic location on a chromosome. When two read pairs are on different chromosomes, those two read pairs are not likely to be linked because the reads came from different chromosomes (i.e., different molecules). Accordingly, an estimate of the spatial null distribution may be generated by this subset of the total sequencing data which indicates read pairs for reads on different chromosome pairs.

Once a null distribution is calculated, the method may proceed to step 1125, by calculating a false positive rate proportional to the product of the percentage of spatial and genomic null links below a threshold spatial and genomic distance. It should be realized that alternative methods for calculating a link quality score may not employ a false positive rate as recited in step 1125, and thus it may be optional. For example, other positive and negative predictive values may be used to test true positive and true negative results. Link quality scores can be derived from a null distribution to assess the reliability or confidence of the observed data. These scores can be based on metrics such as the signal-to-noise ratio, the deviation from the null distribution, or the likelihood of observing the data given the default expectation. Higher link quality scores indicate greater reliability and confidence in the observed results. A statistical test of interest may be performed on the observed data to obtain test statistics or p-values for each expectation (false positive, false negative, true positive, true negative). The test statistics may be calculated on sampled data, where the p-values represent the probability of obtaining test statistics as extreme as, or more extreme than, the observed values under the default expectation. In other examples, a false positive rate may be used as an intermediate metric to estimate a false discovery rate, or any other type of type I error metric.

The method may then proceed to step 1130 by generating a link quality score representing a probability of incorrectly assigning a link between the two polynucleotide reads. The link quality score may be generated based on the calculated genomic null distribution and the calculated spatial null distribution. Calculating the false discovery rate (FDR) from a null distribution may involve determining the proportion of false positives among the total positive findings when no true effects exist. A null distribution may be generated by simulating data under the assumption of no true effects or associations. The disclosure provides methods that may avoid steps of fitting a null distribution or simulating a null distribution by random shuffling of the observed data. As an alternative, the disclosure provides methods that take advantage of complementary information on the genomic sequences (spatial linking data) and may select a subset of that data, which would not present realistic or likely scenarios, to estimate a null distribution and generate a link quality score. Accordingly, systems and methods of the disclosure may advantageously increase the quality and efficiency of data processing associated with polynucleotide sequencing. In particular, practical applications of quantifiable linked reads may include improvements in mapping, aligning, and variant calling.

At step 1135 the method calculates link quality scores for different combinations of thresholds. In some embodiments, the step 1135 may include calculating a link quality score by determining null distributions for each threshold. In some embodiments, step 1135 may include calculating a link quality score by determining a false discovery rate for each threshold. In some embodiments, step 1135 may include calculating a link quality score by determining a prior null distribution that is used for a new threshold. In some embodiments, step 1135 may include generating a link quality score based on a false discovery rate. By utilizing multiple or different combinations of thresholds, different link quality scores can be achieved, thereby allowing for more informed decisions or more accurate placement of linked read pairs. To calculate link quality scores for different combinations of thresholds, various parameters and metrics may be considered such as, the number of linked read pairs, the sequence depth, read overlap, sequence quality, and alignment scores. For each combination of these metrics, a threshold may be established, above or below which a link is considered to meet that particular quality criterion. Then, a link quality score can be calculated based on how many thresholds are met and how well they are met. This may be achieved through weighted averaging, or a composite linking quality score may be generated. This composite linking quality score may be designed to represent the individual scores into a comprehensive metric or series of metrics that encapsulate the overall quality of the links in a sequencing experiment. As an alternative or in addition, the outputs from different thresholds can be used in a hierarchical manner. High-quality connections, as determined by applying the most stringent thresholds, may be used, or analyzed, first. This may be beneficial in applications where computational resources are limited or where there is some benefit in identifying the most reliable links quickly. Lower-quality connections, which meet less stringent thresholds, may be considered later or as supplementary to the high-quality links.

Once the link quality scores have been calculated at the step 1135, the process 1100 can move to a decision step 1140 to determine if a target, or optimal, threshold has been reached. If the target threshold has been reached, then the process 1100 can terminate at an end step. If the target threshold has not been reached, then process 1100 may return to step 1125 so the calculations can be repeated in order to determine a target, or optimal threshold. A predetermined target threshold for identifying genuine connections between read pairs may be set based on prior knowledge from a prior validation. For example, the target threshold might be based on setting a minimum number of linked read pairs or a maximum allowable distance between linked read pairs on a flow cell. An optimal threshold may be determined by post-analysis of previous target thresholds. A threshold may be tailored to maximize specific metrics that may be important for the given task, such as sensitivity, specificity, or a balanced combination of both. Methods such as cross-validation may be used to fine-tune this target threshold in order to minimize errors, including false positive and false-negative linked connections between read pairs.

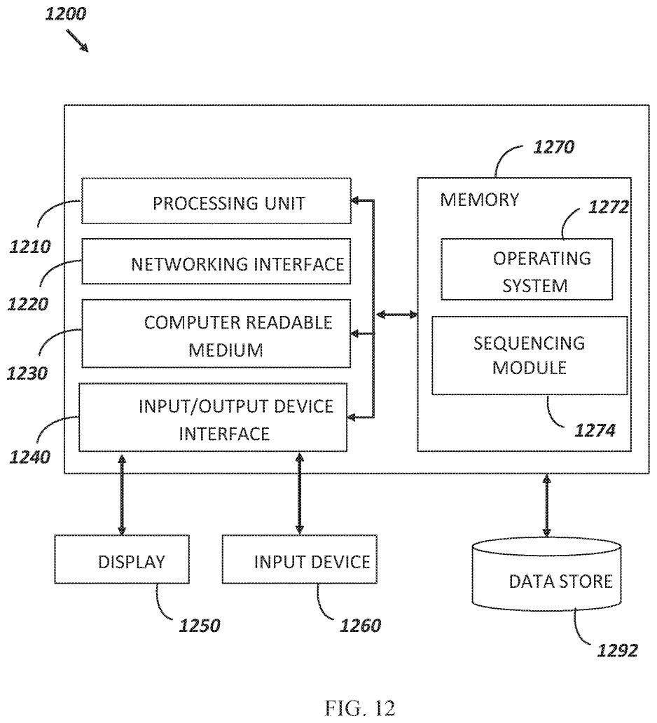

Embodiments of the present disclosure also include a system for analyzing and assembling sequences of polynucleotides. FIG. 12 is a diagram of an exemplary computing system 1200 that may be used in connection with an illustrative sequencing system. The computing system 1200 may be configured to determine a DNA sequence by using the sequencing and assembly methods disclosed herein. The general architecture of the computing system 1200 includes an arrangement of computer hardware and software components. The computing system 1200 may include many more (or fewer) elements. It is not necessary, however, that all of these generally conventional elements be shown in order to provide an enabling disclosure.

As illustrated, the computing system 1200 includes a processing unit 1410, a network interface 1220, a computer-readable medium drive 1230, an input/output device interface 1240, a display 1250, and an input device 1260, all of which may communicate with one another by way of a communication bus. The network interface 1270 may provide connectivity to one or more networks or computing systems. The processing unit 1210 may thus receive information and instructions from other computing systems or services via a network. The processing unit 1210 may also communicate to and from memory 1270 and further provide output information for an optional display 1250 via the input/output device interface 1240. The input/output device interface 1240 may also accept input from the optional input device 1260, such as a keyboard, mouse, digital pen, microphone, touch screen, gesture recognition system, voice recognition system, gamepad, accelerometer, gyroscope, or other input device.

The memory 1270 may contain computer program instructions (grouped as modules or components in some embodiments) that the processing unit 1210 executes in order to implement one or more embodiments. The memory 1270 generally includes RAM, ROM and/or other persistent, auxiliary or non-transitory computer-readable media. The memory 1270 may store an operating system 1272 that provides computer program instructions for use by the processing unit 1210 in the general administration and operation of the computing device 1200. The memory 1270 may further include computer program instructions and other information for implementing aspects of the present disclosure.

For example, in one embodiment, the memory 1270 includes an alignment module 1274 for analyzing and assembling sequences of polynucleotides. The module 1274 can perform the methods disclosed herein, including the method described with respect to the flow diagrams of, for example, FIG. 2. In addition, memory 1270 may include or communicate with the data store 1290 and/or one or more other data stores that store one or more inputs, one or more outputs, and/or one or more results (including intermediate results) of determining a DNA sequence and providing an assembly process according to the present disclosure.

### Systems and Instruments

An aspect of the disclosure is directed to methods for identifying links across an entire genome. In some embodiments, a method may include receiving a BAM file that includes spatial information. The method may proceed by splitting the BAM file into surface and chromosomes. For each subset of the BAM file (surface/chromosome), a “KD-tree” may be constructed, which is a data structure for querying m-dimensional ranges, where m>1. Then, the method may proceed for each point p in each KD-tree t. The KD-tree t may in turn be queried for all points p_neighbors within spatial distance threshold of p. The KD-tree t may be queried for each p2 in p_neighbors. The method may determine a link if p and p2 are within a genomic distance threshold, and then record (p, p2) as a link.

In some embodiments, a method of finding links between read pairs on a flow cell, may include the step of providing sequencing data for read pairs from clusters on the flow cell. The method may also include filtering clusters that are spatially distant from one another, and/or filtering clusters that are genomically distant from one another. The method may include selecting neighboring clusters that are within a spatial distance threshold as neighboring clusters; and the assigning links to two read pairs in the neighboring clusters when the clusters are within a genomic distance threshold. In some embodiments, assigning links to two read pairs may occur when the genomic distance threshold is a preset threshold. In some embodiments, the method may generate a first subset of the sequencing data for read pairs by selecting the clusters that are spatially distant from one another. In some embodiments, the method may include selecting a first cluster that has nucleic acid derived from a first chromosome and a second cluster has nucleic acid from a second, different chromosome.

In some embodiments, the clusters are on the same surface but opposite ends of the flow cell. In some embodiments, the clusters are opposite surfaces of the flow cell. In some embodiments, calculating the spatial null distribution of the plurality of the plurality of read pairs comprises determining read pairs from clusters on the flow cell. In some embodiments, a first cluster has nucleic acid derived from a first chromosome and a second cluster has nucleic acid from a second, different chromosome.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above embodiments may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

In some embodiments, the methods may be written in any of various suitable programming languages, for example compiled languages such as C, C #, C++, Fortran, and Java. Other programming languages could be script languages, such as Perl, MatLab, SAS, SPSS, Python, Ruby, Pascal, Delphi, R and PHP. In some embodiments, the methods are written in C, C #, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly or indirectly held on a computer readable medium and loaded as needed onto a computer system. Further, the methods may be located on computers that are remote to where the data is being produced, such as software found on servers and the like that are maintained in another location relative to where the data is being produced, such as that provided by a third-party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (for example, iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is commonly via Internet connection, either wireless or by a network cable through an access point. In some embodiments, a storage device may be maintained and managed by the individual or entity directly associated with an assay instrument, whereas in other embodiments a storage device may be maintained and managed by a third party, commonly at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, an SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random-access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computers are clustered together to yield a supercomputer network.

In some embodiments, computational methods as described herein are carried out on a collection of inter-or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations. One aspect of the disclosure is directed to a workflow module that may be integrated into existing workflows. In some embodiments, a workflow module may be a two-channel sequencing module and may be integrated into a NGS sequence analysis platform, for example the DRAGEN™ Bio-ID platform from Illumina.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

The terms “polynucleotide,” “oligonucleotide,” “nucleic acid” and “nucleic acid molecules” are used interchangeably herein and refer to a covalently linked sequence of nucleotides of any length (i.e., ribonucleotides for RNA, deoxyribonucleotides for DNA, analogs thereof, or mixtures thereof) in which the 3′ position of the pentose of one nucleotide is joined by a phosphodiester group to the 5′ position of the pentose of the next. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, which is complementary or copy DNA produced from an RNA template, for example by the action of reverse transcriptase. This term refers only to the primary structure of the molecule. Thus, the term includes, without limitation, triple-, double- and single-stranded deoxyribonucleic acid (“DNA”), as well as triple-, double- and single-stranded ribonucleic acid (“RNA”). The nucleotides include sequences of any form of nucleic acid. As apparent from the examples below and elsewhere herein, a nucleic acid can have a naturally occurring nucleic acid structure or a non-naturally occurring nucleic acid analog structure. A nucleic acid can contain phosphodiester bonds; however, in some embodiments, nucleic acids may have other types of backbones, comprising, for example, phosphoramide, phosphorothioate, phosphorodithioate, O-methylphosphoroamidite and peptide nucleic acid backbones and linkages. Nucleic acids can have positive backbones; non-ionic backbones, and non-ribose-based backbones. Nucleic acids may also contain one or more carbocyclic sugars. The nucleic acids used in methods or compositions herein may be single stranded or, alternatively double stranded, as specified. In some embodiments a nucleic acid can contain portions of both double stranded and single stranded sequence, for example, as demonstrated by forked adapters. A nucleic acid can contain any combination of deoxyribo-and ribonucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc. In some embodiments, a nucleic acid can include at least one promiscuous base. A promiscuous base can base-pair with more than one different type of base and can be useful, for example, when included in oligonucleotide primers or inserts that are used for random hybridization in complex nucleic acid samples such as genomic DNA samples. An example of a promiscuous base includes inosine that may pair with adenine, thymine, or cytosine. Other examples include hypoxanthine, 5-nitroindole, acylic 5-nitroindole, 4-nitropyrazole, 4-nitroimidazole and 3-nitropyrrole. Promiscuous bases that can base-pair with at least two, three, four or more types of bases can be used.

As used herein, phrases and/or terms related to probabilities refer to the probability of the fragments of the polynucleotide being located near each other on the flow cell. Relevantly, the probability may be correlated with a distance between the fragments of the polynucleotide in the polynucleotide. In the context of sequencing technologies like those used in next-generation sequencing (NGS), a polynucleotide is a long chain of nucleotides, which can be DNA or RNA. The process typically involves fragmenting these long molecules into smaller pieces for easier sequencing. When these fragments are placed onto a flow cell, which is a glass slide used as a solid surface for sequencing reactions, they are typically spatially distributed in an even and random distribution across the flow cell surface.

The probability that fragments of a polynucleotide are located near each other on the flow cell being correlated with the distance between the fragments in the actual polynucleotide chain refers to the likelihood that segments that are closer together along the chain of the polynucleotide will end up being positioned closer to each other on the flow cell during sequencing. This correlation may be a result of the fragmentation process, or the method used to immobilize the fragments onto the flow cell.

For example, if the fragmentation method merely cuts the polynucleotide randomly and then casting the fragments onto the flow cell, there is a chance that fragments from the same region of the original polynucleotide will be in proximity on the flow cell. However, if the fragments are attached to the flow cell in a random fashion, then the initial proximity in the polynucleotide may not be preserved. On the other hand, certain methods, such as those according to the disclosure, may retain some spatial information, leading to a higher probability that closely located sequences in the polynucleotide remain close on the flow cell. The relevance of this correlation depends on the sequencing technology used. In some technologies, preserving the relative positions of the fragments can be beneficial for reconstructing the original sequence of the polynucleotide, as it can aid in the mapping and assembly process. Here, “Spatially co-located” in the context of genomic fragments refers to the occurrence of two or more DNA fragments being positioned in close physical proximity to one another in a given space. This term is used to describe how fragments are situated relative to each other on the flow cell during sequencing.

As used herein, the term “fragment,” when used in reference to a first nucleic acid, is intended to mean a second nucleic acid having a part or portion of the sequence of the first nucleic acid. Generally, the fragment and the first nucleic acid are separate molecules. The fragment can be derived, for example, by physical removal from the larger nucleic acid, by replication or amplification of a region of the larger nucleic acid, by degradation of other portions of the larger nucleic acid, a combination thereof or the like. The term can be used analogously to describe sequence data or other representations of nucleic acids. As used herein, the term “haplotype” refers to a set of alleles at more than one locus inherited by an individual from one of its parents. A haplotype can include two or more loci from all or part of a chromosome. Alleles include, for example, single nucleotide polymorphisms (SNPs), short tandem repeats (STRs), gene sequences, chromosomal insertions, chromosomal deletions etc. The term “phased alleles” refers to the distribution of the particular alleles from a particular chromosome, or portion thereof. Accordingly, the “phase” of two alleles can refer to a characterization or representation of the relative location of two or more alleles on one or more chromosomes.

As used herein, the term “active region” or “region of interest” refers to a segment of the genome that is specifically targeted for sequencing or currently being analyzed during a sequencing method step. These regions may be a single region or a window covering multiple sequence reads at a time. When it comes to methods of assembly or structural variant detection, an active region is often the focal point where advanced sequencing techniques are applied to obtain a highly accurate sequence. In the context of structural variant detection, active regions may be scrutinized using specialized techniques that can detect larger-scale genomic alterations, such as inversions, translocations, or large indels. These variants may not be evident with typical sequencing approaches and often require methods like paired-end or long-read sequencing to span the entire region of interest. This is also relevant for assembling a genome from scratch, where active regions may be targeted for individual steps of a sequencing process to be sequenced with a higher coverage depth or with longer reads to ensure that these important parts of the genome are assembled correctly.

As used herein, the term “Anchor Read” refers to reads that can be mapped with high confidence or unambiguously to unique positions in a genome. Anchor reads serve as reliable reference points in the mapping process, providing high-confidence alignments between the sequence reads and the reference genome. These anchor reads are usually characterized by a high degree of similarity to known sequences in the reference genome, often facilitated by processes that assign high-quality alignment scores based on the number of matches, mismatches, gaps, and other criteria.

As used herein, the term “flanking” genomic sequencing refers to stretches of DNA or RNA fragments that are situated at a certain distance from a specific region of interest, such as an anchor read, a gene, a mutation site, or a repetitive element. These regions may be used as reference points and may not necessarily be directly next to the region of interest. The distance between the flanking region and the target can vary widely, from just a few base pairs to several kilobases away, depending on the genome and the method of used to link reads to anchor reads. For example, some methods of the disclosure are able to link reads from several kilobases away and may be even more sensitive to structural variants that are several kilobases long.

In the context of anchor sequence reads, as described above, flanking regions serve as reference points for alignment but are not required to be immediately adjacent to the sequence of interest. An anchor read may include sequences that are several hundred or even thousands of base pairs away from the flanking regions. These non-adjacent flanking regions are particularly useful when the anchor read includes repetitive sequences that occur frequently in the genome, or in identifying structural variants. By identifying unique flanking sequences at a distance, methods according to the disclosure can still map the anchor read to the correct location on the genome.

The use of distant flanking regions is a useful strategy of the disclosure for use in genomic sequencing to achieve accurate mapping. It allows for the unambiguous alignment of reads that would otherwise be difficult to place due to the presence of repetitive or complex sequences. By considering a range of distances for potential flanking regions, various tools can effectively ‘anchor’ reads to their proper location in the genome, which is useful for reliable genome assembly and the accurate identification of genetic variants.

As used herein, the term “unambiguous mapping,” in the context of genomic sequencing refers to the process of correctly and uniquely assigning a sequenced DNA fragment to a single location in a reference genome. This means that the sequence of the fragment is so distinctive that it matches one and only one region in the reference genome with a high degree of confidence. By way of example, challenges in mapping may arise because genomes often contain repetitive sequences. If a fragment comes from a repetitive region, it may map to multiple locations, leading to ambiguous mapping. Ambiguity in mapping can complicate genetic analyses and may lead to incorrect conclusions. Therefore, the goal is to achieve unambiguous mapping wherever possible, which is more likely with longer reads, longer synthetic reads, long sequences of linked reads, or with fragments that include unique sequences flanking repetitive regions.

As used herein, the term “ambiguous mapping,” or “ambiguously mapping” refers to a scenario when a fragment of DNA or RNA (a sequence of nucleotides) aligns with two or more locations in the target polynucleotide sequence with low confidence and/or a similar level of confidence for the two or more locations. When sequencing a genome, individual fragments are generated and then will usually be matched back to a reference genome to determine their original location. This process is known as mapping. If a read comes from a unique sequence in the genome, the read can be mapped unambiguously. However, if the read is derived from a sequence that is, for example, repeated in the genome, a mapping process may find multiple potential origins for the read. These multiple matching locations make it unclear where the read actually came from, hence the term “ambiguous mapping”.

As used herein, the term “alignment field” refers to a category of data within an alignment record, specifically detailing the relationship between a sequence read and a reference sequence. These alignment records are generally stored in standard formats like the Sequence Alignment/Map (SAM) file, which is widely used for storing sequence alignment data. The SAM format organizes alignment information into several predefined fields, each field representing a specific aspect of the alignment. For instance, fields such as QNAME (query name), FLAG (alignment properties), RNAME (reference sequence name), and POS (position of alignment) are standard components of an alignment record. Additional fields include MAPQ (mapping quality), indicating the confidence in the alignment, and CIGAR (Compact Idiosyncratic Gapped Alignment Report), which succinctly characterizes how the read aligns to the reference, encompassing matches, mismatches, insertions, and deletions.

As described herein, alignment fields are useful for interpreting the alignment's quality and accuracy. These fields contain information such as the precise (or approximate) starting position of the alignment on the reference sequence, the sequence of the read itself, the quality scores for each base in the read, and details about the read's mate in paired-end sequencing. For example, a CIGAR string is useful in identifying mismatches and gaps that may suggest variations between the read and the reference.

As described herein, an alignment field can also indicate an ambiguous alignment if, for example, the MAPQ score is low, which signifies that the read aligns equally well to multiple locations in the reference genome. Another indication of ambiguity can be inferred from the FLAG field, which may denote whether a read is mapped in a proper pair or not. Reads not properly paired often result from one read of a pair mapping confidently to one location while its mate maps to another, or not at all. In cases where the reference genome contains repetitive sequences, a read derived from such a region might map to several locations with similar scores, leading to ambiguous alignment. Ambiguously aligned reads may be flagged and optionally excluded from further analysis.

As used herein, the term “baseline scenario” (particularly when it involves the use of truth data sets), refers to a set of sequence data that has been validated and is used as a comparative standard for assessing the quality of sequencing efforts. The size of the sequence data may vary from a short sequence to a long sequence up to the size of a reference genome. Baseline scenarios may be generated for a section of the sequencing data set and used as a comparison for the rest of the same sequencing data set. For example, a portion of the sequencing data may be evaluated for some metric, such as sequence depth, and used to determine if the rest of the sequencing data (or a portion thereof) is abnormal and indicates some genomic variant.

Truth data sets may include sequences with known variants, including single nucleotide polymorphisms (SNPs), insertions, deletions, and other genetic features that have been verified through rigorous testing and are considered highly accurate. These truth sets may be employed as benchmarks to evaluate how well a new sequencing run can identify and replicate known genetic variations. They provide a point of comparison to determine the error rate of the new sequencing process by highlighting discrepancies between the newly sequenced data and the validated sequences.

As used herein, the term “putative” generally refers to “generally considered or reputed to be,” which implies an assumption based on some evidence, but without conclusive proof. In the context of genomics, when referring to “putative structural variants,” the term suggests that these are structural changes in the genome—such as deletions, duplications, insertions, inversions, or translocations—that have been identified as possible or likely variations from the reference genome but have not yet been fully validated. Putative structural variants are typically identified through computational analyses of genomic data as described herein. Methods according to the disclosure can predict these variants by analyzing patterns in sequencing data that suggest deviations from the expected alignment to a reference genome. For instance, reads, or sets of linked reads, that span breakpoint junctions of an inversion, or clusters of reads that indicate a duplication, might lead to the identification of putative structural variants. However, these predictions may require further investigation to determine their validity.

As used herein, the term “threshold distance” in the context of identifying structural variants in a polynucleotide refers to a predefined maximum/minimum distance within which sequence reads must fall relative to anchor sequence reads to be considered relevant, such as, for example, relevant as part of the same structural variant event. The use of threshold distances is useful for filtering out less relevant reads when analyzing high-throughput sequencing data to detect genomic rearrangements such as deletions, insertions, duplications, inversions, or translocations.

As described above, anchor sequence reads are those that can be aligned with high confidence to a known location on the reference genome. In the vicinity of these anchor reads, other reads that do not align with high MAPQ (e.g. MAPQ=60) may still be informative for variant detection if they are within a certain proximity—a threshold distance. The range of threshold distances can vary depending on the type of structural variant being investigated and the sequencing technology used. For example, for small Indels (Insertions/Deletions), the threshold distance might be quite small, often in the range of a few bases up to 50 bases, as the changes are relatively close to the anchor reads. For larger structural variants, the threshold distance may be set from a few hundred to several thousand bases. The larger the expected variant, the greater the distance that might be considered. When parts of the chromosome have been rearranged significantly, the threshold distance could be very large, spanning tens to hundreds of thousands of bases, as the reads indicating the breakpoints of such events could be far from the anchor points in the linear genome sequence.

These threshold distances may or may not be arbitrary. The thresholds may be determined based on empirical evidence and statistical models that account for the distribution of reads and the expected frequency of sequencing errors or natural genomic variation. By setting appropriate threshold distances, researchers can minimize false positives (incorrectly calling a variant where there is none) and false negatives (failing to detect an actual variant). The threshold distance as disclosed herein is a useful parameter in bioinformatics pipelines for structural variant detection, balancing sensitivity (detecting true variants) and specificity (not calling false variants).

Note that in the context of spatially linked reads, distance may refer to genomic distance or a physical distance in the flow cell. As used in the disclosure, the term distance may refer to both (e.g., a threshold distance is applied to both genomic distance and physical distance) and/or may be understood in the context to refer to one or the other type of distance.

As used herein, genomic distance refers to the number of base pairs between two points on a sequence within a genome. The genomic distance is a linear measurement that considers the sequence length alone, irrespective of a polynucleotide's three-dimensional structure. For example, if one gene starts at position 100,000 and another gene starts at position 300,000 on a chromosome, the genomic distance between them is 200,000 base pairs. As describe herein, in the context of identifying structural variants, a threshold genomic distance may be set to determine how far apart two reads can be to still be considered as potentially related to the same structural variant. If two reads are within this threshold genomic distance, they may be analyzed together to identify potential deletions, insertions, or other variants.

Similarly, the term “physical distance,” refers to the actual space between two fragments of polynucleotide a flow cell. This distance may reflect the way DNA is fragmented on the flow cell. When applying thresholds to physical distances, researchers are often looking at the interaction between DNA segments in a three-dimensional space, such as in chromosome conformation capture experiments (e.g., Hi-C). A threshold for physical distance may be used to determine whether two DNA fragments are close enough to each other in order to have originated from the same original polynucleotide sequence.

Thresholds for both genomic and physical distances are useful for interpreting complex genomic data. For genomic distances, thresholds may be applied as described herein, in sequence alignment and variant calling processes to decide whether reads should be considered together for variant detection. For instance, in paired-end sequencing, if the distance between two reads exceeds the expected genomic distance based on the insert size, this could indicate a potential deletion or insertion.

For physical distances, thresholds are used in analyzing links between fragments of polynucleotides. Here, thresholds can help identify fragments that are spatially collocated (such as, by example, within a physical distance threshold) more or less frequently than expected versus random chance.

As used herein, the phrase “located spatially close” refers to the proximity of sequence fragments relative to each other or within a given space. In a broad sense, it means that the fragments are near each other in terms of physical distance, which can be measured in units, such as nanometers or units of distance on a flow cell. Defining what is considered “close” is context dependent. Close may be defined by a threshold distance, which sets a cutoff for how near two points should be to be considered spatially close. Close may also refer generally to distance, such as determining how close two fragments are to each other, and not necessarily imply close proximity.

As used herein, the phrase “Spatially linked read pairs” in the context of genomic sequencing refers to pairs of DNA sequence reads that originate from the same polynucleotide sequence and are expected to be a certain distance apart based on, for example, the size of the fragments. These read pairs are considered ‘linked’ because they would have been physically connected in the genome before the DNA is fragmented during, for example, library preparation for sequencing.

When determining which sequence reads are linked to other reads, such as anchor sequence reads, spatially linked read pairs are very useful. As described above, an anchor sequence read is a read that has been confidently mapped to a specific location on the reference genome. By looking at the spatially linked pair of a read, researchers can infer where the other fragment should map to the genome. If the second read of the pair does not map where expected (based on the known length of the DNA fragment), this may suggest the presence of a structural variant between the two reads.

Detecting structural variants, which include insertions, deletions, inversions, and translocations, often poses challenges because they inherently involve larger, more complex alterations to the genome than single-nucleotide polymorphisms (SNPs) or small indels. The high-confidence anchor reads become particularly crucial in this context. When reads are mapped to a reference genome, some may align perfectly or nearly perfectly, serving as anchor reads, while others may not align well or may align to multiple locations. These less reliably mapped reads may in fact be indicative of structural variants, and their accurate mapping often relies on the context provided by anchor reads.

For example, in the case of a deletion in the sample genome relative to the reference genome, an anchor read may align well at one end but have a ‘dangling’ other end that doesn't align anywhere in proximity. The presence of a high-confidence anchor read can provide the context needed to recognize that the ‘dangling’ end is not a sequencing error or artifact but is likely part of a structural variant. Likewise, for insertions, translocations, or inversions, anchor reads can offer the stable framework within which the unusual or less confidently mapped reads can be understood.

In paired-end sequencing, one read in the pair might serve as the anchor read while the other spans a structural variant. The anchor read assures that the pair exists in a specific region, giving bioinformaticians confidence to explore what the other read in the pair might reveal about structural changes in the genome. Tools specialized in detecting structural variants often use these anchor reads as starting points for ‘walking’ along the genome to find the boundaries of structural variants.

As used herein, the term “nucleotide sequence” is intended to refer to the order and type of nucleotide monomers in a nucleic acid polymer. A nucleotide sequence is a characteristic of a nucleic acid molecule and can be represented in any of a variety of formats including, for example, a depiction, image, electronic medium, series of symbols, series of numbers, series of letters, series of colors, etc. The information can be represented, for example, at single nucleotide resolution, at higher resolution (e.g., indicating molecular structure for nucleotide subunits) or at lower resolution (e.g., indicating chromosomal regions, such as haplotype steps). A series of “A,” “T,” “G,” and “C” letters is a well-known sequence representation for DNA that can be correlated, at single nucleotide resolution, with the actual sequence of a DNA molecule. A similar representation is used for RNA except that “T” is replaced with “U” in the series.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zennor, silica or silica-based materials including silicon and modified silicon, carbon, metals, inorganic glasses, optical fiber bundles, and polymers. Particularly useful solid supports for some embodiments are located within a flow cell apparatus. Exemplary flow cells are set forth in further detail below.

As used herein, the term “flow cell” is intended to mean a chamber having a surface across which one or more fluid reagents can be flowed. Generally, a flow cell will have an ingress opening and an egress opening to facilitate the flow of fluid. A flow cell can have multiple surfaces. Examples of flow cells and related fluidic systems and detection platforms that can be readily used in the methods of the present disclosure are described, for example, in Bentley et al, Nature 456:53-59 (2008), WO 04/018497; U.S. Pat. No. 7,057,026; WO 91/06678; WO 07/123744; U.S. Pat. Nos. 7,429,492; 7,311,414; 7,415,019; 7,405,381, and US 2008/0108082, each of which is incorporated herein by reference.

In many embodiments, a solid support to which nucleic acids are attached in a method set forth herein will have a continuous or monolithic surface. Thus, fragments can attach at spatially random locations wherein the distance between nearest neighbor fragments (or nearest neighbor clusters derived from the fragments) will be variable. The resulting arrays will have a variable or random spatial pattern of features. Alternatively, a solid support used in a method set forth herein can include an array of features that are present in a repeating pattern. In such embodiments, the features provide the locations to which modified nucleic acid polymers, or fragments thereof, can attach. Particularly useful repeating patterns are hexagonal patterns, rectilinear patterns, grid patterns, patterns having reflective symmetry, patterns having rotational symmetry, or the like. The features to which a modified nucleic acid polymer, or fragment thereof, attach can each have an area that is smaller than about 1 mm², 500 μm², 100 μm², 25 μm², 10 μm², 4 μm², 1 μm², 500 nm², or 100 nm². Alternatively, or additionally, each feature can have an area that is larger than about 100 nm², 350 nm², 500 nm², 1 μm², 2.5 μm², 5 μm², 10 μm², 100 μm², or 500 μm². A cluster or colony of nucleic acids that result from amplification of fragments on an array (whether patterned or spatially random) can similarly have an area that is in a range above or between an upper and lower limit selected from those exemplified above.

For embodiments that include an array of features on a surface, the features can be discrete, being separated by interstitial regions. Alternatively, some or all of the features on a surface can be abutting (i.e., not separated by interstitial regions). Whether the features are discrete or abutting, the average size of the features and/or average distance between the features can vary such that arrays can be high density, medium density or lower density. High density arrays are characterized as having features with average pitch of less than about 15 μm. Medium density arrays have average feature pitch of about 15 to 30 μm, while low density arrays have average feature pitch of greater than 30 μm. An array useful in the invention can have feature pitch of, for example, less than 100 μm, 50 μm, 10 μm, 5 μm, 1 μm or 0.5 μm. Alternatively, or additionally, the feature pitch can be, for example, greater than 0.1 μm, 0.5 μm, 1 μm, 5 μm, 10 μm, 50 μm, or 100 μm.

As used herein, the term “source” is intended to include an origin for a nucleic acid molecule, such as a tissue, cell, organelle, compartment, or organism. The term can be used to identify or distinguish an origin for a particular nucleic acid in a mixture that includes origins for several other nucleic acids. A source can be a particular organism in a metagenomic sample having several different species of organisms. In some embodiments the source will be identified as an individual origin (e.g., an individual cell or organism). Alternatively, the source can be identified as a species that encompasses several individuals of the same type in a sample (e.g., a species of bacteria or other organism in a metagenomic sample having several individual members of the species along with members of other species as well).

As used herein, the term “surface,” when used in reference to a material, is intended to mean an external part or external layer of the material. The surface can be in contact with another material such as a gas, liquid, gel, polymer, organic polymer, second surface of a similar or different material, metal, or coat. The surface, or regions thereof, can be substantially flat. The surface can have surface features such as wells, pits, channels, ridges, raised regions, pegs, posts or the like. The material can be, for example, a solid support, gel, or the like.

As an example, in some embodiments, fragments derived from a long nucleic acid molecule captured at the surface of a flow cell occur in a line across the surface of the flow cell (e.g., if the nucleic acid was stretched out prior to fragmentation or amplification) or in a cloud on the surface. Further, a physical map of the immobilized nucleic acid can then be generated. The physical map thus correlates the physical relationship of clusters after immobilized nucleic acid is amplified. Specifically, the physical map is used to calculate the probability that sequence data obtained from any two clusters are linked, as described in the incorporated materials of WO 2012/025250. Alternatively, or additionally, the physical map can be indicative of the genome of a particular organism in a metagenomic sample. In this latter case the physical map can indicate the order of sequence fragments in the organism's genome; however, the order need not be specified and instead the mere presence of two or more fragments in a common organism (or other source or origin) can be sufficient basis for a physical map that characterizes a mixed sample and one or more organisms therein. In some embodiments, a polynucleotide may be provided from a sample comprising a plurality of polynucleotides from at least two organisms. In some embodiments, methods may include determining linked sequence reads relating to an individual organism. In some embodiments, methods may include performing a metagenomic analysis on the plurality of polynucleotides.

In some embodiments, the physical map is generated by imaging the solid support to establish the location of the immobilized nucleic acid molecules across the surface. In some embodiments, the immobilized nucleic acid is imaged by adding an imaging agent to the solid support and detecting a signal from the imaging agent. In some embodiments, the imaging agent is a detectable label. Suitable detectable labels, include, but are not limited to, protons, haptens, radionuclides, enzymes, fluorescent labels, chemiluminescent labels, and/or chromogenic agents. For example, in some embodiments, the imaging agent is an intercalating dye or non-intercalating DNA binding agent. Any suitable intercalating dye or non-intercalating DNA binding agent as are known in the art can be used, including, but not limited to those set forth in U.S. 2012/0282617, which is incorporated herein by reference.

In certain embodiments, a plurality of modified nucleic acid molecules is flowed onto a flow cell comprising a plurality of nano-channels. As used herein, the term nano-channel refers to a narrow channel into which a long linear nucleic acid molecule is stretched. In some embodiments, no more than 1, 2, 3, 4, 5, 6, 7, 8, 9, 10, 11, 12, 13, 14, 15, 16, 17, 18, 19, 20, 30, 40, 50, 60 70, 80, 90, 100, 300, 400, 400, 500, 600, 700, 1400, 900 or no more than 500 individual long strands of nucleic acid are stretched across each nano-channel. In some embodiments the individual nano-channels are separated by a physical barrier that prevents individual long strands of target nucleic acid from interacting with multiple nano-channels. In some embodiments, the solid support comprises at least 10, 50, 100, 300, 500, 500, 3000, 5000, 10000, 30000, 50000, 80000 or at least 100000 nano-channels.

As used herein, the term “target,” when used in reference to a nucleic acid polymer, is intended to linguistically distinguish the nucleic acid, for example, from other nucleic acids, modified forms of the nucleic acid, fragments of the nucleic acid, and the like. Any of a variety of nucleic acids set forth herein can be identified as target nucleic acids, examples of which include genomic DNA (gDNA), messenger RNA (mRNA), copy or complimentary DNA (cDNA), and derivatives or analogs of these nucleic acids.

As used herein, the term “transposase” is intended to mean an enzyme that is capable of forming a functional complex with a transposon element-containing composition (e.g., transposons, transposon ends, transposon end compositions) and catalyzing insertion or transposition of the transposon element-containing composition into a target DNA with which it is incubated, for example, in an in vitro transposition reaction. The term can also include integrases from retrotransposons and retroviruses. Transposases, transposomes and transposome complexes are generally known to those of skill in the art, as exemplified by the disclosure of US Pat. App. Pub. No. 2010/0120098, which is incorporated herein by reference. Although many embodiments described herein refer to Tn5 transposase and/or hyperactive Tn5 transposase, it will be appreciated that any transposition system that is capable of inserting a transposon element with sufficient efficiency to tag a target nucleic acid can be used. In particular embodiments, a preferred transposition system is capable of inserting the transposon element in a random or in an almost random manner to tag the target nucleic acid. As used herein, the term “transposome” is intended to mean a transposase enzyme bound to a nucleic acid. typically, the nucleic acid is double stranded. For example, the complex can be the product of incubating a transposase enzyme with double-stranded transposon DNA under conditions that support non-covalent complex formation. Transposon DNA can include, without limitation, Tn5 DNA, a portion of Tn5 DNA, a transposon element composition, a mixture of transposon element compositions or other nucleic acids capable of interacting with a transposase such as the hyperactive Tn5 transposase.

As used herein, the term “transposon element” is intended to mean a nucleic acid molecule, or portion thereof, that includes the nucleotide sequences that form a transposome with a transposase or integrase enzyme. typically, the nucleic acid molecule is a double stranded DNA molecule. In some embodiments, a transposon element is capable of forming a functional complex with the transposase in a transposition reaction. As non-limiting examples, transposon elements can include the 19-bp outer end (“OE”) transposon end, inner end (“IE”) transposon end, or “mosaic end” (“ME”) transposon end recognized by a wild-type or mutant In5 transposase, or the R1 and R2 transposon end as set forth in the disclosure of US Pat. App. Pub. No. 2010/0120098, which is incorporated herein by reference. Transposon elements can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands.

A typical NGS sequencing run yields millions of short sequences that are eventually mapped on a reference genome. A percentage of good-quality reads (1-5%) are discarded because of ambiguous genomic location. Increasing read length (2×500 or long-read sequencing), designing a specialized process to map reads on specific regions of the genome (targeted callers), using expensive and time-consuming library preparation (Illumina CLR), or a combination thereof may be implemented to address the need for disambiguating such reads that would normally be discarded. However, such approaches are costly, laborious, and time intensive. Spatial information (X and Y coordinates) obtained from a solid support surface) can be leveraged to identify fragments that are generated from a single long input fragment and subsequentially be used to improve mapping reads in ambiguous positions.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or step diagrams of methods, apparatus (systems), and computer program products according to embodiments of the disclosure. It will be understood that each step of the flowchart illustrations and/or step diagrams, and combinations of steps in the flowchart illustrations and/or step diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or step diagram step or steps. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or step diagram(s) step or steps.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or step diagram step or steps. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and step diagrams in the Figures illustrate the architecture, functionality, and operation of possible implementations of systems, methods, and computer program products according to various embodiments of the present disclosure. In this regard, each step in the flowchart or step diagrams may represent a service, module, segment, or portion of instructions, which comprises one or more executable instructions for implementing the specified logical function(s). In some alternative implementations, the functions noted in the steps may occur out of the order noted in the Figures. For example, two steps shown in succession may, in fact, be executed substantially concurrently, or the steps may sometimes be executed in the reverse order, depending upon the functionality involved. In addition, certain steps may be omitted in some implementations. The methods and processes described herein are also not limited to any particular sequence, and the steps or states relating thereto can be performed in other sequences that are appropriate.

It will also be noted that each step of the step diagrams and/or flowchart illustration, and combinations of steps in the step diagrams and/or flowchart illustration, can be implemented by special purpose hardware-based systems that perform the specified functions or acts or carry out combinations of special purpose hardware and computer instructions. For example, any of the processes, methods, elements, steps, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above embodiments may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only and are not intended to limit the scope of the disclosure. Indeed, the novel methods described herein may be embodied in a variety of other forms. Furthermore, various omissions, substitutions and changes in the methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method of generating a link quality score, comprising:
calculating a genomic null distribution of a plurality of read pairs on a flow cell having approximately zero probability of being linked with one another;
calculating a spatial null distribution of a plurality of read pairs having approximately zero probability of being linked with one another; and
generating a link quality score representing a probability of incorrectly assigning a link between two polynucleotide reads in the plurality of read pairs based on the calculated genomic null distribution and the calculated spatial null distribution.

2. The method of claim 1, wherein calculating the genomic null distribution of the plurality of read pairs comprises determining read pairs from clusters of nucleic acid sequences on the flow cell.

3. The method of claim 2, wherein the clusters are spatially distant from one another on the same or opposite surfaces of a flow cell.

4. The method of claim 3, wherein the clusters are on the same surface but opposite ends of the flow cell.

5. The method of claim 3, wherein the clusters are on opposite surfaces of the flow cell.

6. The method of claim 1, wherein calculating the spatial null distribution of the plurality of read pairs comprises determining the nucleic acid sequence of read pairs from clusters on the flow cell.

7. The method of claim 6, wherein a first cluster has a nucleic acid sequence derived from a first chromosome and a second cluster has a nucleic acid sequence from a second, different chromosome.

8. The method of claim 1, further comprising applying threshold levels which limit the spatial distance and genomic distance between the plurality of read pairs on the flow cell.

9. The method of claim 1, further comprising determining at least one threshold level such that read pairs derive from the same DNA molecule with a predetermined probability for spatial distance and genomic distance using the quality of the link.

10. The method of claim 8, further comprising estimating a linking rate for the total links between read pairs on the flow cell for the threshold levels.

11. The method of claim 1, further comprising selecting a threshold level based on the link quality score.

12. A system for sequencing a polynucleotide comprising:
at least one processor; and
a processor configured to perform a method, comprising:
retrieving data comprising polynucleotide sequence reads and their spatial location on a flow cell, wherein the flow cell comprises clusters of amplified nucleic acid fragments of a polynucleotide, and wherein a probability of the clusters being located near each other on the flow cell is correlated with a genomic distance between the nucleic acid fragments of the polynucleotide and wherein each cluster provides one or more of the sequence reads;
determining links between sequence reads originating from the same polynucleotide, wherein a link between sequence reads is based on at least one of:
(a) the linked sequence reads have a link quality score of at least 20 on a Phred scale, and the linked sequence coverage has an average depth of at least 30-fold (30×) of a reference genome;
(b) the percentage of sequence reads on the flow cell with a determined link to at least one other sequence read is greater than 45% and the average sequence coverage is at least 70× coverage for all sequence reads;
(c) the percentage of sequence reads on the flow cell with a determined link to at least one other sequence read is greater than 35%, and the average sequence coverage is at least 90× coverage for all sequence reads; and
(d) the percentage of sequence reads on the flow cell with a determined link to at least one other sequence read is greater than 25% and the average sequence coverage is at least 120× coverage for all sequence reads.

13. A method for sequencing a polynucleotide, comprising:
obtaining sequence reads from a flow cell, wherein the flow cell comprises clusters of amplified nucleic acid fragments of the polynucleotide, and wherein a probability of the clusters being located near each other on the flow cell is correlated with a genomic distance between the nucleic acid fragments of the polynucleotide and wherein each cluster provides one or more of the sequence reads; and
determining links between sequence reads originating from the same polynucleotide molecule, wherein at least one of:
(a) the linked sequence reads have a link quality score of at least 20 on a Phred scale, and the linked sequence coverage has an average depth of at least 30-fold (30×) coverage of a reference genome;
(b) the percentage of sequence reads on the flow cell with a determined link to at least one other sequence read is greater than 45%, and the average sequence coverage is at least 70× coverage for all sequence reads;
(c) the percentage of sequence reads on the flow cell with a determined link to at least one other sequence read is greater than 35%, and the average sequence coverage is at least 90× coverage for all sequence reads; and
(d) the percentage of sequence reads on the flow cell with a determined link to at least one other sequence read is greater than 25%, and the average sequence coverage is at least 120× coverage for all sequence reads.

14. The method of claim 13, wherein obtaining sequence reads from the flow cell comprises using a polynucleotide concentration of less than 10 picomolar in a loading sample on the flow cell.

15. The method of claim 13, wherein obtaining sequence reads from the flow cell comprises loading less than 1 microgram of polynucleotides in a loading sample on the flow cell.

16. The method of claim 13, wherein obtaining sequence reads from the flow cell comprises loading polynucleotides with at least 60,000 base pairs in a loading sample on the flow cell.

17. The method of claim 13, wherein obtaining sequence reads from the flow cell comprises using a flow rate of less than 250 μL/min for the flow of solution across the flow cell during sample loading on the flow cell.

18. The method of claim 13, wherein obtaining sequence reads from the flow cell comprises using fewer than three purification steps on a loading sample prior to sample loading onto the flow cell.

19. The method of claim 13, wherein obtaining sequence reads from the flow cell comprises loading a crude lysate loading sample onto the flow cell, wherein the crude lysate sample has been lysed but not extracted.

20. The method of claim 13, wherein obtaining sequence reads from the flow cell comprises sequencing the polynucleotides after two or more loading cycles on the flow cell.

21.-23. (canceled)
